(19) United States
(12) Patent Application Publication
(10) Pub. No.: US 2009/0318423 A1
(43) Pub. Date: Dec. 24, 2009

(54) Ion Channel Modulators & Uses Thereof

(75) Inventors: Geoff Lawton, Cambridgeshire (GB); Roland Kozlowski, Cambridgeshire (GB); Dayle Hogg, Cambridgeshire (GB)

Correspondence Address: Sunstein Kann Murphy & Timbers LLP, 125 SUMMER STREET, BOSTON, MA, 02110-1618, US

(73) Assignee: LECTUS THERAPEUTICS LIMITED, Cambridge. Cambridgeshire (GB)

(21) Appl. No.: 12/443,402

(22) PCT Filed: Sep. 28, 2007
(86) PCT No.: PCT/GB2007/050593

§ 371 Date: Apr. 14, 2009

(30) Foreign Application Priority Data
Sep. 29, 2006 (GB) 0619176.1

(51) Int. Cl. A61K 31/55; C07D 223/16; A61K 31/472; C07D 217/26

(52) U.S. Cl. 514/217.01; 540/594; 514/310; 546/143

(57) ABSTRACT

Compounds of general formula (1) and pharmacologically acceptable salts and prodrugs thereof: Formula (1) wherein A and B are CH₂ or CH₂CH₂, R1 is hydrogen, alkyl, cycloalkyl, aryl, aralkyl or heteroaralkyl, R2, R3 and R4 are selected from hydrogen, alkyl, halogen, haloalkyl, alkoxy, alkoxycarbonyl, carboxyl, hydroxyl or cyano; X is R5CO, R5SO₂, R5R7NCO, R5R7NSO₂, R5SO₂NR7CO or CO₂R8, Y is R6CO, R6SO₂, R6R7NCO, R6R7NSO₂, R6SO₂NR7CO or CO₂R8, R5 and R6 are hydrogen, alkyl, aryl, aralkyl, heteroaryl or heteroaralkyl, R7 is hydrogen, alkyl, aryl or aralkyl, and R8 is alkyl, aryl, aralkyl, alkoxyalkyl, heteroaryl or heteroarylalkyl, are of use in the prophylaxis or treatment of diseases and conditions in which Kv1.x channels are involved, such as lower urinary tract disorders, cardiovascular diseases and pain.

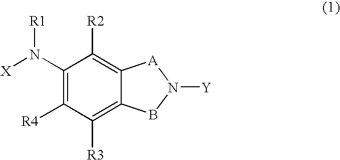

### FIELD OF THE INVENTION

The present invention relates to ion channel modulators, and more particularly to heterocyclic compounds which inhibit the interaction between the pore-forming (alpha) subunits of Kv1 voltage-gated potassium channels and accessory (Kvbeta subunit) proteins.

### BACKGROUND TO THE INVENTION

Voltage-dependent potassium (Kv) channels conduct potassium ions across cell membranes in response to changes in the membrane voltage and thereby can regulate cellular excitability by modulating (increasing or decreasing) the electrical activity of the cell.

Mammalian homologues (Kv1.1-Kv1.8) of so called Kv1 potassium channel alpha subunits encoded by the Drosophila Shaker gene can form tetrameric protein complexes that span the plasma membrane of cells and allow the passage of K+ ions. These tetrameric protein complexes of Kv1.x channels constitute the ion channel pore-forming domain.

Functional Kv1 channels consist of a tetramer of transmembrane spanning Kv1.x channel subunits may be associated with and regulated by accessory (Kvbeta) proteins that are able to modulate the function of ion channel pore-forming domains (for reviews, see: Xu, J. & Li, M., Trends Cardiovasc. Med., 1998, 8, 229-234; Pongs, O., et al., Ann. N.Y. Acad. Sci., 1999, 868, 344-355).

Functional Kv1 channels can exist as multimeric structures formed by the association of either identical or dissimilar Kv1.x and/or Kvbeta proteins. Modulation of functional Kv1 channel complexes by Kvbeta subunits is believed to be Kv subfamily specific (Sewing et al, Neuron 1996, 15, 455-463).

Kvbeta subunits bind to Kv1.x channel alpha subunits through an interaction domain known as the ‘T1 domain’ or ‘tetramerisation domain’ located on the N-terminus of Kv1.x channel alpha subunits. The T1 domain was originally identified as an amino-terminal fragment of Kv1.x channels that was necessary for alpha subunit assembly (Li et alt, Science, 1992, 257, 1225-1229; Shen et al., Neuron, 1993, 11, 67-76). The putative sequence of the interaction site between Kv1.x alpha subunits with Kvbeta subunits has been determined and a sequence identified within the so-called T1 domain that is necessary for this interaction (Yu et al., Neuron, 1996, 16, 441-453; Sewing et al. Neuron 1996, 15, 455-463). More recently, elucidation of the crystal structure of the interaction between the Kv1.1 T1 domain and the Kvbeta2 subunit, has established the current understanding of sequence of the critical interaction site between Kv1.1 channels and Kvbeta subunits (Gulbis et al, Science, 2000, 289, 123-127).

“Kv1.x” channels consist of at least 8 members which include one or more of the following mammalian channels. Kv1.1, Kv1.2, Kv1.3, Kv1.4, Kv1.5, Kv1.6, Kv1.7, Kv1.8 and any mammalian or non-mammalian equivalents or variants (including splice variants) thereof.

“Kvbeta” proteins may include one or more of the following mammalian subunits: Kvbeta1.1, Kvbeta1.2, Kvbeta1.3, Kvbeta2.1, Kvbeta2.2, Kvbeta3, Kvbeta3.1, Kvbeta4 and any mammalian or non-mammalian equivalents or variants (including splice variants) thereof.

At a functional level, interactions between Kv1.x channels and Kvbeta proteins can confer modulation (increasing or decreasing) of a number of features of functional Kv1 channels including, but not limited to (i) the transport or chaperone of Kv1.x channels to the plasma membrane of a given cell type (e.g. Shi et al., Neuron, 1996, 16, 843-852) and/or (ii) gating properties such as channel inactivation (for example see Rettig et al., Nature, 1994, 369, 289-294; Heinemann et al., Journal of Physiology, 1996, 493, 625-633; Bähring et al., Molecular Membrane Biology, 1994, 21, 19-25).

In addition to modulation of Kv channel “Inactivation”, which refers to the closing of the Kv channel by any mechanism such as by N-type inactivation, Kvbeta subunits can also exert effects on other gating properties (see Hille, B. Ionic Channels of Excitable Membranes, Sunderland, M A, 1992) by mechanisms which may alter the time and voltage dependency of the open (conducting state), closed (non-conducting state) and inactivated states (non-conducting state) of Kv1.x channels.

Furthermore, the interaction between specific Kvbeta subunits and different Kv1.x channel compositions may confer differential modulation to Kv1.x channel currents (Bähring et al., Molecular Membrane Biology, 1994, 21, 19-25). This phenomenon may account for the wide diversity of K+ channels. However, exact subunit compositions of native K+ channels and the physiologic role that particular channels play are, in most cases, still unclear.

Compounds which inhibit the interaction between Kv1.x channels and Kvbeta proteins and thus preserve or enhance either the conducting state of Kv1.x channels (e.g. though reducing the rate of Kv1.x channel inactivation) and/or increase the transport of Kv1.x channels to the plasma membrane, are defined as “Kv1.x channel openers” (Kv1.x channel opening).

Compounds which inhibit the interaction between Kv1.x channels and Kvbeta proteins and thus preserve either the closed state of Kv1.x channels and/or decrease the transport of Kv1.x channels to the plasma membrane, are defined as “Kv1.x channel inhibitors” (Kv1.x channel inhibition).

Such Kv1.x channel openers or Kv1.x channel inhibitors have potential utility in the treatment, prevention, inhibition, amelioration or alleviation of symptoms of a number of conditions or disease states including:

“Lower Urinary Tract Disorders”. Lower urinary tract disorders is intended to encompass both painful (any lower urinary tract disorder involving sensations or symptoms that a patient subjectively describes as producing or resulting in pain) and non-painful lower urinary tract disorders (any lower urinary tract disorder involving sensations or symptoms, including mild or general discomfort, that is subjectively described as not producing or resulting in pain). “Lower urinary tract disorders” also includes any lower urinary tract disorder characterised by overactive bladder with and/or without loss of urine, urinary frequency, urinary urgency, and nocturia. Thus, lower urinary tract disorders includes overactive bladder or overactive urinary bladder (including, overactive detrusor, detrusor instability, detrusor hyperreflexia, sensory urgency and the symptoms of detrusor overactivity), urge incontinence or urinary urge incontinence, stress incontinence or urinary stress incontinence, lower urinary tract symptoms including obstructive urinary symptoms such as slow urination, dribbling at the end of urination, inability to urinate and/or the need to strain to urinate at an acceptable rate or irritate symptoms such as frequency and/or urgency. Lower urinary tract disorders may also include neurogenic bladder that occurs as the result of neurological damage due to disorders including but not limited to stroke, Parkinson's disease, diabetes, multiple sclerosis, peripheral neuropathy, or spinal cord lesions. Lower urinary tract disorders may also include prostatitis, interstitial cystitis, benign prostatic hyperplasia, and, in spinal cord injured patients, spastic bladder.

“Anxiety and Anxiety-Related Conditions”. Anxiety and anxiety related conditions is intended to include, but is not limited to, anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias. Specific anxiety related phobias include, but are not limited to, fear of animals, insects, storms, driving, flying, heights or crossing bridges, closed or narrow spaces, water; blood or injury, as well as extreme fear of inoculations or other invasive medical or dental procedures.

“Epilepsy”. Epilepsy is intended to include, but is not limited to, one or more of the following seizures: simple partial seizures, complex partial seizures, secondary generalised seizures, generalised seizures including absence seizures, myoclonic seizures, clonic seizures, tonic seizures, tonic clonic seizures and atonic seizures.

“Pain Disorders”. Pain is intended to include but is not limited to one or more on the following: acute pain such as musculoskeletal pain, post operative pain and surgical pain; chronic pain such as chronic inflammatory pain (e.g. rheumatoid arthritis and osteoarthritis), neuropathic pain (e.g. post herpetic neuralgia, trigeminal neuralgia and sympathetically maintained pain) and pain associated with cancer and fibromyalgia; pain associated with migraine, pain (both chronic and acute), and/or fever and/or inflammation of conditions such as rheumatic fever, symptoms associated with influenza or other viral infections, such as the common cold; lower back and neck pain; headache; toothache; sprains and strains; myositis; neuralgia; synovitis; arthritis, including rheumatoid arthritis; degenerative joint diseases, including osteoarthritis; gout and ankylosing spondylitis; tendinitis; bursitis; skin-related conditions, such as psoriasis, eczema, burns and dermatitis; injuries, such as sports injuries and those arising from surgical and dental procedures.

“Gynaecological Pain”, for example, dysmenorrhoea, labour pain and pain associated with endometriosis.

“Cardiac Arrhythmias”. Cardiac arrhythmias includes but is not limited to atrial fibrillation, atrial flutter, atrial arrhythmia and supaventricular tachycardia.

“Thromboembolic Events” such as stroke.

“Cardiovascular Diseases” such as angina pectoris, hypertension and congestive heart failure.

“Disorders of the Auditory System” such as tinnitus.

“Migraine”.

“Gastrointestinal Disorders” including reflux esauphagitis, functional dispepsia, motility disorders (including constipation and diarrhea), and irritable bowel syndrome.

“Vascular and Visceral Smooth Muscle Disorders” including asthma, pulmonary hypertension, chronic obstructive pulmonary disease, adult respiratory distress syndrome, peripheral vascular disease (including intermittent claudication), venous insufficiency, impotence, cerebral and coronary spasm and Raynaud's disease.

“Cell Proliferative Disorders” including restenosis and cancer (including leukemia), treating or preventing gliomas including those of lower and higher malignancy.

“Metabolic Disorders” such as diabetes (including diabetic retinopathy, diabetic nephropathy and diabetic neuropathy), insulin resistance/insensitivity and obesity.

“Memory Loss” including Alzheimer's disease and dementia.

Other “CNS-Mediated Motor Dysfunction Disorders” including Parkinson's disease and ataxia.

“Ophthalmic Disorders” such as ocular hypertension.

Thus, it would be desirable to identify Kv1.x channel openers and Kv1.x channel inhibitors for the prophylaxis or treatment of a number of disease states including lower urinary tract disorders and pain indications. Using assays based on the interaction between Kv1.x channel T1 domains and Kvbeta subunits immobilised through an affinity tag, we have discovered a new family of heterocyclic compounds which inhibit the interaction between Kv1.x channels and Kvbeta proteins.

### DESCRIPTION OF THE INVENTION

In a first aspect of the present invention, there is provided a compound represented by the general formula (1a) or a pharmacologically acceptable salt or prodrug thereof:

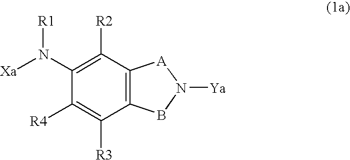

wherein:
A and B are independently CH₂ or CH₂CH₂;
R1 is a hydrogen atom, an alkyl group, a cycloalkyl group, an aryl group, an aralkyl group or a heteroaralkyl group;
R2, R3 and R4 are independently selected from hydrogen atoms, alkyl groups, halogen atoms, haloalkyl groups, alkoxy groups, alkoxycarbonyl groups, carboxyl groups, hydroxyl groups, nitro groups, amino groups, monoalkylamino groups, dialkylamino groups, acylamino groups, alkoxycarbonylamino groups, alkylsulphonyl groups, arylsulphonyl groups, aminosulphonyl groups and cyano groups;
Xa is R5aR7NSO₂ or R5aSO₂NR7CO, wherein R5a and R7 are as defined below;
Ya is selected from R6CO, R6SO₂, R6R7NCO, R6R7NSO₂, R6SO₂NR7CO and CO₂R8, wherein R6, R7 and R8 are as defined below;
R5a and R6 are independently selected from hydrogen atoms, alkyl groups, aryl groups, aralkyl groups, heteroaryl groups and heteroaralkyl groups,
R7 is a hydrogen atom, an alkyl group, an aryl group or an aralkyl group;
R8 is an alkyl group, an aryl group, an aralkyl group, an alkoxyalkyl group, a heteroaryl group or a heteroarylalkyl group.

In a second aspect of the present invention, there is provided a compound represented by the general formula (1b) or a pharmacologically acceptable salt or prodrug thereof:

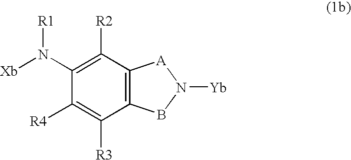

wherein:
A and B are independently CH₂ or CH₂CH₂;
R1 is a hydrogen atom, an alkyl group, a cycloalkyl group, an aryl group, an aralkyl group or a heteroaralkyl group;
R2, R3 and R4 are independently selected from hydrogen atoms, alkyl groups, halogen atoms, haloalkyl groups, alkoxy groups, alkoxycarbonyl groups, carboxyl groups, hydroxyl groups, nitro groups, amino groups, monoalkylamino groups, dialkylamino groups, acylamino groups, alkoxycarbonylamino groups, alkylsulphonyl groups, arylsulphonyl groups, aminosulphonyl groups and cyano groups;
Xb is R5bCO, wherein R5b is a hydrogen atom, an alkyl group having from 1 to 4 carbon atoms or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
Yb is selected R6SO₂, R6R7NSO₂, R6SO₂NR7CO, wherein R6 and R7 are as defined below;
R6 is selected from hydrogen atoms, alkyl groups, aryl groups, aralkyl groups, heteroaryl groups and heteroaralkyl groups;
R7 is a hydrogen atom, an alkyl group, an aryl group or an aralkyl group.

In a third aspect of the present invention, there is provided a compound represented by the general formula (1c) or a pharmacologically acceptable salt or prodrug thereof:

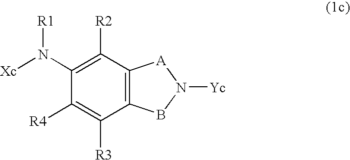

wherein:
A and B are independently CH₂ or CH₂CH₂;
R1 is a hydrogen atom, an alkyl group, a cycloalkyl group, an aryl group, an aralkyl group or a heteroaralkyl group;
R2, R3 and R4 are independently selected from hydrogen atoms, alkyl groups, halogen atoms, haloalkyl groups, alkoxy groups, alkoxycarbonyl groups, carboxyl groups, hydroxyl groups, nitro groups, amino groups, monoalkylamino groups, dialkylamino groups, acylamino groups, alkoxycarbonylamino groups, alkylsulphonyl groups, arylsulphonyl groups, aminosulphonyl groups and cyano groups;
Xc is R5cSO₂, wherein R5c is an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
Yc is selected from R6CO, R6SO₂, R6R7NSO₂ and R6SO₂NR7CO, wherein R6 and R7 are as defined below;
R6 is selected from hydrogen atoms, alkyl groups, aryl groups, aralkyl groups, heteroaryl groups and heteroaralkyl groups;
R7 is a hydrogen atom, an alkyl group, an aryl group or an aralkyl group.

In a fourth aspect of the present invention, there is provided a compound represented by the general formula (1d) or a pharmacologically acceptable salt or prodrug thereof:

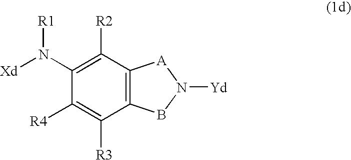

wherein:
A and B are independently CH₂ or CH₂CH₂;
R1 is a hydrogen atom, an alkyl group, a cycloalkyl group, an aryl group, an aralkyl group or a heteroaralkyl group;
R2, R3 and R4 are independently selected from hydrogen atoms, alkyl groups, halogen atoms, haloalkyl groups, alkoxy groups, alkoxycarbonyl groups, carboxyl groups, hydroxyl groups, nitro groups, amino groups, monoalkylamino groups, dialkylamino groups, acylamino groups, alkoxycarbonylamino groups, alkylsulphonyl groups, arylsulphonyl groups, aminosulphonyl groups and cyano groups;
Xd is CO₂R8, wherein R8 is as defined below;
Yd is selected from R6CO, R6SO₂, R6R7NCO, R6R7NSO₂ and
R6SO₂NR7CO, wherein R6 and R7 are as defined below,
R6 is selected from hydrogen atoms, alkyl groups,
aryl groups, aralkyl groups, heteroaryl groups and heteroaralkyl groups;
R7 is a hydrogen atom, an alkyl group, an aryl group or an aralkyl group;
R8 is an alkyl group, an aryl group, an aralkyl group, an alkoxyalkyl group, a heteroaryl group or a heteroarylalkyl group.

Preferred compounds of the present invention include:

(5) compounds according to any one of (1) to (4) and pharmacologically acceptable salts and prodrugs thereof wherein R1 is a hydrogen atom, an alkyl group having from 1 to 6 carbon atoms or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups;
(6) compounds according to (1) to (4) and pharmacologically acceptable salts and prodrugs thereof wherein R1 is a hydrogen atom or an alkyl group having from 1 to 4 carbon atoms;
(7) compounds according to (1) to (4) and pharmacologically acceptable salts and prodrugs thereof wherein R1 is a hydrogen atom;
(8) compounds according to any one of (1) to (4) and pharmacologically acceptable salts and prodrugs thereof wherein R2, R3 and R4 are independently selected from hydrogen atoms, alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups substituted with an alkoxy group having from 1 to 6 carbon atoms, carboxy groups, hydroxyl groups and cyano groups;
(9) compounds according to any one of (1) to (4) and pharmacologically acceptable salts and prodrugs thereof wherein R2, R3 and R4 are independently selected from hydrogen atoms, alkyl groups having from 1 to 4 carbon atoms and halogen atoms;
(10) compounds according to any one of (1) to (4) and pharmacologically acceptable salts and prodrugs thereof wherein R2, R3 and R4 are independently selected from hydrogen atoms, methyl groups, ethyl groups, fluorine atoms and chlorine atoms;
(11) compounds according to any one of (1) to (4) and pharmacologically acceptable salts and prodrugs thereof wherein each of R2, R3 and R4 is a hydrogen atom;
(12) compounds according to any one of (1) and (5) to (11) and pharmacologically acceptable salts and prodrugs thereof wherein Xa is a group of formula R5aSO₂R7NCO or R5aR7NSO₂, wherein
R5a is: a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms, and
R7 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups;
(13) compounds according to any one of (1) and (5) to (11) and pharmacologically acceptable salts and prodrugs thereof wherein Xa is a group of formula R5aSO₂R7NCO or R5aR7NSO₂, wherein
R5a is an aryl group having from 6 to 10 carbon atoms which may optionally be substituted with at least one substituent selected from alkyl groups having from 1 to 4 carbon atoms, halogen atoms, alkyoxy groups having from 1 to 4 carbon atoms, phenyl groups and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, and R7 is a hydrogen atom or an alkyl group having from 1 to 4 carbon atoms;
(14) compounds according to any one of (1) and (5) to (11) and pharmacologically acceptable salts and prodrugs thereof wherein Xa is a group of formula R5aSO₂R7NCO, wherein R5a is a phenyl group which may optionally be substituted with a methyl group, a t-butyl group, a fluorine atom, a chlorine atom, a methoxy group or a phenyl group and R7 is a hydrogen atom;
(15) compounds according to any one of (1) and (5) to (11) and pharmacologically acceptable salts and prodrugs thereof wherein Xa is a group of formula R5aR7NSO₂, wherein R5a and R7 each represent a hydrogen atom;
(16) compounds according to any one of (2) and (5) to (11) and pharmacologically acceptable salts and prodrugs thereof wherein Xb is a group of formula R5bCO, wherein R5b is: a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms;
(17) compounds according to any one of (2) and (5) to (11) and pharmacologically acceptable salts and prodrugs thereof wherein Xb is a group of formula R5bCO, wherein R5b is a hydrogen atom; an alkyl group having from 1 to 4 carbon atoms or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
(18) compounds according to any one of (2) and (5) to (11) and pharmacologically acceptable salts and prodrugs thereof wherein Xb is a group of formula R5bCO, wherein R5b is a hydrogen atom, a methyl group, a benzyl group or a phenethyl group;
(19) compounds according to any one of (3) and (5) to (11) and pharmacologically acceptable salts and prodrugs thereof wherein Xc is a group of formula R5cSO₂, wherein R5c is: a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms;
(20) compounds according to any one of (3) and (5) to (11) and pharmacologically acceptable salts and prodrugs thereof wherein Xc is a group of formula R5cSO₂, wherein R5c is an alkyl group having from 1 to 4 carbon atoms or an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
(21) compounds according to any one of (3) and (5) to (11) and pharmacologically acceptable salts and prodrugs thereof wherein Xc is a group of formula R5cSO₂, wherein R5c is a phenyl group which may optionally be substituted with at least one substituent selected from methyl groups, ethyl groups, chlorine atoms and fluorine atoms;
(22) compounds according to any one of (4) to (5) to (11) and pharmacologically acceptable salts and prodrugs thereof wherein X is a group of formula CO₂R8 wherein R8 is: an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups;
(23) compounds according to any one of (4) and (5) to (11) and pharmacologically acceptable salts and prodrugs thereof wherein Xd is a group of formula CO₂R8 wherein R8 is an alkyl group having from 1 to 4 carbon atoms, an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
(24) compounds according to any one of (1), (3) to (15) and (19) to (23) and pharmacologically acceptable salts and prodrugs thereof wherein Ya, Yc or Yd is a group of formula R6CO, wherein R6 is a hydrogen atom, an alkyl group having from 1 to 6 carbon atoms; an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms;
(25) compounds according to any one of (1), (3) to (15) and (19) to (23) and pharmacologically acceptable salts and prodrugs thereof wherein Ya, Yc or Yd is a group of formula R6CO, wherein R6 is a hydrogen atom, an alkyl group having from 1 to 4 carbon atoms or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
(26) compounds according to any one of (1), (3) to (15) and (19) to (23) and pharmacologically acceptable salts and prodrugs thereof wherein Ya, Yc or Yd is a group of formula R6CO, wherein R6 is a hydrogen atom; a methyl group, a benzyl group or a phenethyl group;
(27) compounds according to any one of (1) to (23) and pharmacologically acceptable salts and prodrugs thereof wherein Ya, Yb, Yc or Yd is a group of formula R6SO₂, wherein R6 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms;
(28) compounds according to any one of (1) to (23) and pharmacologically acceptable salts and prodrugs thereof wherein Ya, Yb, Yc or Yd is a group of formula R6SO₂, wherein R6 is an alkyl group having from 1 to 4 carbon atoms or an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
(29) compounds according to any one of (1) to (23) and pharmacologically acceptable salts and prodrugs thereof wherein Ya, Yb, Yc or Yd is a group of formula R6SO₂, wherein R6 is a phenyl group which may optionally be substituted with at least one substituent selected from methyl groups, ethyl groups, chlorine atoms and fluorine atoms;
(30) compounds according to any one of (1), (4) to (15), (22) and (23) and pharmacologically acceptable salts and prodrugs thereof wherein Ya or Yd is a group of formula R6R7NCO, wherein
R6 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms, and
R7 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups;
(31) compounds according to any one of (1), (4) to (15), (22) and (23) and pharmacologically acceptable salts and prodrugs thereof wherein Ya or Yd is a group of formula R6R7NCO, wherein
R6 is a hydrogen atom, an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, and
R7 is a hydrogen atom or an alkyl group having from 1 to 4 carbon atoms,
(32) compounds according to any one of (1), (4) to (15), (22) and (23) and pharmacologically acceptable salts and prodrugs thereof wherein Ya or Yd is a group of formula R6R7NCO, wherein
R6 is a hydrogen atom, a phenyl group which may optionally be substituted with at least one substituent selected from methyl groups, ethyl groups, fluorine atoms and chlorine atoms, or a benzyl group which may optionally be substituted with at least one substituent selected from methyl groups, ethyl groups, fluorine atoms and chlorine atoms, and
R7 is a hydrogen atom, a methyl group or an ethyl group;
(33) compounds according to any one of (1), (4) to (15), (22) and (23) and pharmacologically acceptable salts and prodrugs thereof wherein Ya or Yd is a group of formula R6R7NCO, wherein R6 is a hydrogen atom, a benzyl group or a phenyl group which is optionally substituted with a methyl group, and R7 is a hydrogen atom;
(34) compounds according to any one of (1) to (23) and pharmacologically acceptable salts and prodrugs thereof wherein Ya, Yb, Yc or Yd is a group of formula R6SO₂R7NCO, wherein
R6 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms, and
R7 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups;
(35) compounds according to any one of (1) to (23) and pharmacologically acceptable salts and prodrugs thereof wherein Ya, Yb, Yc or Yd is a group of formula R6SO₂R7NCO, wherein
R6 is an aryl group having from 6 to 10 carbon atoms which may optionally be substituted with at least one substituent selected from alkyl groups having from 1 to 4 carbon atoms, halogen atoms, alkoxy groups having from 1 to 4 carbon atoms and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms; and
R7 is a hydrogen atom or an alkyl group having from 1 to 4 carbon atoms;
(36) compounds according to any one of (1) to (23) and pharmacologically acceptable salts and prodrugs thereof wherein Ya, Yb, Yc or Yd is a group of formula R6SO₂R7NCO, wherein R6 is a phenyl group which may optionally be substituted with a methyl group, a t-butyl group, a chlorine atom, a fluorine atom or a methoxy group, and R7 is a hydrogen atom;
(37) compounds according to any one of (1) and (5) to (15) and pharmacologically acceptable salts and prodrugs thereof wherein Ya is a group of formula CO₂R8 wherein R8 is an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups;
(38) compounds according to any one of (1) and (5) to (15) and pharmacologically acceptable salts and prodrugs thereof. Ya is a group of formula CO₂R8 wherein R8 is an alkyl group having from 1 to 4 carbon atoms, an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
(39) compounds according to (1) and pharmacologically acceptable salts and prodrugs thereof wherein:
each of R1, R2, R3 and R4 is a hydrogen atom;
Xa is a group of formula R5aSO₂R7NCO, wherein R5a is a phenyl group which may optionally be substituted with a methyl group, a t-butyl group, a fluorine atom, a chlorine atom, a methoxy group or a phenyl group and R7 is a hydrogen atom; and
Ya is a group of formula R6CO (wherein R6 is a hydrogen atom; a methyl group, a benzyl group or a phenethyl group), a group of formula R6SO₂ (wherein R6 is a phenyl group which may optionally be substituted with at least one substituent selected from methyl groups, ethyl groups, chlorine atoms and fluorine atoms) or a group of formula R6R7NCO (wherein R5 is a hydrogen atom, a benzyl group or a phenyl group which is optionally substituted with a methyl group and R7 is a hydrogen atom);
   (40) compounds according to (2) and pharmacologically acceptable salts and prodrugs thereof wherein:
each of R1, R2, R3 and R4 is a hydrogen atom;
Xb is a group of formula R5bCO, wherein R5b is a hydrogen atom, a methyl group, a benzyl group or a phenethyl group; and
Yb is a group of formula R6bSO₂R7NCO, wherein R6b is a phenyl group which may optionally be substituted with a methyl group, a t-butyl group, a fluorine atom, a chlorine atom or a methoxy group and R7 is a hydrogen atom;
   (41) compounds according to (3) and pharmacologically acceptable salts and prodrugs thereof wherein:
each of R1, R2, R3 and R4 is a hydrogen atom;
Xc is a group of formula R5SO₂ (wherein R5 is a phenyl group which may optionally be substituted with at least one substituent selected from methyl groups, ethyl groups, chlorine atoms and fluorine atoms); and
Yc is a group of formula R6SO₂R7NCO (wherein R6 is a phenyl group which may optionally be substituted with a methyl group, a t-butyl group, a fluorine atom, a chlorine atom or a methoxy group and R7 is a hydrogen atom);
   (42) compounds according to (4) and pharmacologically acceptable salts and prodrugs thereof wherein:
each of R1, R2, R3 and R4 is a hydrogen atom;
Xd is a group of formula CO₂R8 wherein R8 is an alkyl group having from 1 to 4 carbon atoms, an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms; and
Yc is a group of formula R6SO₂R7NCO (wherein R6 is a phenyl group which may optionally be substituted with a methyl group, a t-butyl group, a fluorine atom, a chlorine atom, a methoxy group or a phenyl group and R7 is a hydrogen atom);
   (43) compounds according to (1) and pharmacologically acceptable salts and prodrugs thereof wherein:
each of R1, R2, R3 and R4 is a hydrogen atom;
Ya is a group of formula R6CO, wherein R6 is a hydrogen atom, a methyl group, a benzyl group or a phenethyl group; and
Xa is a group of formula R5aSO₂R7NCO, wherein R5a is a phenyl group which may optionally be substituted with a methyl group, a t-butyl group, a fluorine atom, a chlorine atom, a methoxy group or a phenyl group and R7 is a hydrogen atom;
   (44) compounds according to (1) and pharmacologically acceptable salts and prodrugs thereof wherein:
each of R1, R2, R3 and R4 is a hydrogen atom;
Ya is a group of formula R6SO₂ (wherein R6 is a phenyl group which may optionally be substituted with at least one substituent selected from methyl groups, ethyl groups, chlorine atoms and fluorine atoms); and
Xa is a group of formula R5aSO₂R7NCO (wherein R5a is a phenyl group which may optionally be substituted with a methyl group, a t-butyl group, a fluorine atom, a chlorine atom, a methoxy group or a phenyl group and R7 is a hydrogen atom);
   (45) compounds according to (1) and pharmacologically acceptable salts and prodrugs thereof wherein:
each of R1, R2, R3 and R4 is a hydrogen atom;
Ya is a group of formula R6R7NCO (wherein R6 is a hydrogen atom, a benzyl group or a phenyl group which is optionally substituted with a methyl group, and R7 is a hydrogen atom); and
Xa is a group of formula R5aSO₂R7NCO (wherein R5a is a phenyl group which may optionally be substituted with a methyl group, a t-butyl group, a fluorine atom, a chlorine atom, a methoxy group or a phenyl group and R7 is a hydrogen atom);
   (46) compounds according to (2) and pharmacologically acceptable salts and prodrugs thereof wherein:
each of R1, R2, R3 and R4 is a hydrogen atom;
Yb is a group of formula R6SO₂R7NCO, wherein R6 is a phenyl group which may optionally be substituted with a methyl group, and R7 is a hydrogen atom; and either
Xb is a group of formula R5bCO (wherein R5b is a hydrogen atom; a methyl group, a benzyl group or a phenethyl group), or
Xc is a group of formula R5cSO₂ (wherein R5c is a phenyl group which may optionally be substituted with at least one substituent selected from methyl groups, ethyl groups, chlorine atoms and fluorine atoms); and
   (47) a compound according to (1) to (4) or a pharmacologically acceptable salt or prodrug thereof selected from the group consisting of:
N-phenylsulfonyl-7-[({[(4-methylphenyl)sulfonyl]amino}carbonyl)amino]-1,2,4,5-tetrahydro-3H-3-benzazepine-3-carboxamide;
N-acetyl-7-[({[(4-methylphenyl)sulfonyl]amino}carbonyl)amino]-1,2,4,5-tetrahydro-3H-3-benzazepine-3-carboxamide;
N-benzylaminocarbonyl-7-[({[(4-methylphenyl)sulfonyl]amino}-carbonyl)amino]-1,2,4,5-tetrahydro-3H-3-benzazepine-3-carboxamide;
N-(1-oxo-3-phenyl-propanyl)-7-[({[(4-methylphenyl)sulfonyl]amino}-carbonyl)amino]-1,2,4,5-tetrahydro-3H-3-benzazepine-3-carboxamide;
N-formyl-7-[({[(4-methylphenyl)sulfonyl]amino}carbonyl)amino]-1,2,4,5-tetrahydro-3H-3-benzazepine-3-carboxamide;
N-[(4-methylphenyl)sulfonyl]-7-phenylsulfonylamino-1,2,4,5-tetrahydro-3H-3-benzazepine-3-carboxamide;
N-[(4-methylphenyl)sulfonyl]-7-acetylamino-1,2,4,5-tetrahydro-3H-3-benzazepine-3-carboxamide;
N-[(4-methylphenyl)sulfonyl]-7-benzylaminocarbonylamino-1,2,4,5-tetrahydro-3H-3-benzazepine-3-carboxamide;
N-[(4-methylphenyl)sulfonyl]-7-(1-oxo-3-phenyl-propanylamino)-1,2,4,5-tetrahydro-3H-3-benzazepine-3-carboxamide;
N-[(4-methylphenyl)sulfonyl]-7-formylamino-1,2,4,5-tetrahydro-3H-3-benzazepine-3-carboxamide;
7-[({[(4-methylphenyl)sulfonyl]amino}carbonyl)amino]-1,2,4,5-tetrahydro-3H-3-benzazepine-3-carboxamide;
N-phenylsulfonyl-7-phenylsulfonylaminocarbonylamino]-1,2,4,5-tetrahydro-3H-3-benzazepine-3-carboxamide;
N-[(4-methylphenyl)sulfonyl]-6-({[(4-methylphenyl)sulfonyl]-carbamoyl}amino)-3,4-dihydroisoquinoline-2(1H)-carboxamide;
N-[(4-methylphenyl)sulfonyl]-5-[({[(4-methylphenyl)sulfonyl]amino}carbonyl)amino]-1,3-dihydro-2H-isoindole-2-carboxamide;
N-[(4-methylphenyl)sulfonyl]-7-({[(4-methylphenyl)sulfonyl]carbamoyl}amino)-1,2,4,5-tetrahydro-3H-3-benzazepine-3-carboxamide;
N-phenylsulfonyl-7-{[(phenylsulfonyl)carbamoyl]amino}-1,2,4,5-tetrahydro-3H-3-benzazepine-3-carboxamide;
N-[(3-methylphenyl)sulfonyl]-7-({[(3-methylphenyl)sulfonyl]carbamoyl}amino)-1,2,4,5-tetrahydro-3H-3-benzazepine-3-carboxamide;
N-[(2-chlorophenyl)sulfonyl]-7-({[(2-chlorophenyl)sulfonyl]carbamoyl}amino)-1,2,4,5-tetrahydro-3H-3-benzazepine-3-carboxamide;
N-[(4-chlorophenyl)sulfonyl]-7-({[(4-chlorophenyl)sulfonyl]carbamoyl}amino)-1,2,4,5-tetrahydro-3H-3-benzazepine-3-carboxamide;
N-[(4-fluorophenyl)sulfonyl]-7-({[(4-fluorophenyl)sulfonyl]carbamoyl}amino)-1,2,4,5-tetrahydro-3H-3-benzazepine-3-carboxamide;
N-[(4-methoxyphenyl)sulfonyl]-7-({[(4-methoxyphenyl)sulfonyl]carbamoyl}amino)-1,2,4,5-tetrahydro-3H-3-benzazepine-3-carboxamide;
N-[(4-tert-butylphenyl)sulfonyl]-7-({[(4-tert-butylphenyl)sulfonyl]carbamoyl}amino)-1,2,4,5-tetrahydro-3H-3-benzazepine-3-carboxamide;
N-[(4-methylphenyl)sulfonyl]-7-({[(4-methylphenyl)sulfonyl]carbamoyl}amino)-3,4-dihydroisoquinoline-2(1H)-carboxamide;
N-[(4-methylphenyl)sulfonyl]-7-([(4-methylphenyl)sulfonyl]amino)-1,2,4,5-tetrahydro-3H-3-benzazepine-3-carboxamide;
N-[(4-methylphenyl)sulfonyl]-7-([(4-biphenyl)sulfonyl]amino)-1,2,4,5-tetrahydro-3H-3-benzazepine-3-carboxamide; and
N-[(4-methylphenyl)sulfonyl]-7-([(4-methylphenyl)phenyl)sulfonyl]-amino)-1,2,4,5-tetrahydro-3H-3-benzazepine.

In a second aspect of the present invention, there is provided a pharmaceutical composition comprising a pharmacologically acceptable diluent or carrier and an active ingredient, wherein said active ingredient is a compound according to any one of (1) to (47) or a pharmacologically acceptable salt or prodrug thereof as an active ingredient thereof.

In a third aspect of the present invention, there is provided a compound according to any one of (1) to (47) or a pharmacologically acceptable salt or prodrug thereof for use as a medicament.

In a fourth aspect of the present invention, there is provided use of a compound according to any one of (1) to (47) or a pharmacologically acceptable salt or prodrug thereof in the preparation of a medicament for the prophylaxis or treatment of a disease in which Kv1.x channels are involved.

In a fifth aspect of the present invention, there is provided use of a compound according to any one of (1) to (47) or a pharmacologically acceptable salt or prodrug thereof in the preparation of a medicament for the prophylaxis or treatment of a condition or disease ameliorated by Kv1.x channel opening.

In a sixth aspect of the present invention, there is provided use of a compound according to any one of (1) to (47) or a pharmacologically acceptable salt or prodrug thereof in the preparation of a medicament for the prophylaxis or treatment of a condition or disease ameliorated by Kv1.x channel inhibition.

In a seventh aspect of the present invention, there is provided use of a compound according to any one of (1) to (47) or a pharmacologically acceptable salt or prodrug thereof in the preparation of a medicament for the prophylaxis or treatment of Lower Urinary Tract Disorders.

In an eighth aspect of the present invention, there is provided use of a compound according to any one of (1) to (47) or a pharmacologically acceptable salt or prodrug thereof in the preparation of a medicament for the prophylaxis or treatment of Anxiety and Anxiety-Related Conditions.

In a ninth aspect of the present invention, there is provided use of a compound according to any one of (1) to (47) or a pharmacologically acceptable salt or prodrug thereof in the preparation of a medicament for the prophylaxis or treatment of Epilepsy.

In a tenth aspect of the present invention, there is provided use of a compound according to any one of (1) to (47) or a pharmacologically acceptable salt or prodrug thereof in the preparation of a medicament for the prophylaxis or treatment of Pain Disorders.

In a eleventh aspect of the present invention, there is provided use of a compound according to any one of (1) to (47) or a pharmacologically acceptable salt or prodrug thereof in the preparation of a medicament for the prophylaxis or treatment of Gynaecological Pain.

In a twelfth aspect of the present invention, there is provided use of a compound according to any one of (1) to (47) or a pharmacologically acceptable salt or prodrug thereof in the preparation of a medicament for the prophylaxis or treatment of Cardiac Arrhythmias.

In a thirteenth aspect of the present invention, there is provided use of a compound according to any one of (1) to (47) or a pharmacologically acceptable salt or prodrug thereof in the preparation of a medicament for the prophylaxis or treatment of Thromboembolic Events.

In a fourteenth aspect of the present invention, there is provided use of a compound according to any one of (1) to (47) or a pharmacologically acceptable salt or prodrug thereof in the preparation of a medicament for the prophylaxis or treatment of Cardiovascular Diseases.

In a fifteenth aspect of the present invention, there is provided use of a compound according to any one of (1) to (47) or a pharmacologically acceptable salt or prodrug thereof in the preparation of a medicament for the prophylaxis or treatment of Disorders of the Auditory System.

In a sixteenth aspect of the present invention, there is provided use of a compound according to any one of (1) to (47) or a pharmacologically acceptable salt or prodrug thereof in the preparation of a medicament for the prophylaxis or treatment of Migraine.

In an seventeenth aspect of the present invention, there is provided use of a compound according to any one of (1) to (47) or a pharmacologically acceptable salt or prodrug thereof in the preparation of a medicament for the prophylaxis or treatment of Gastrointestinal Disorders.

In a eighteenth aspect of the present invention, there is provided use of a compound according to any one of (1) to (47) or a pharmacologically acceptable salt or prodrug thereof in the preparation of a medicament for the prophylaxis or treatment of Vascular and Visceral Smooth Muscle Disorders.

In a nineteenth aspect of the present invention, there is provided use of a compound according to any one of (1) to (47) or a pharmacologically acceptable salt or prodrug thereof in the preparation of a medicament for the prophylaxis or treatment of Cell Proliferative Disorders.

In a twentieth aspect of the present invention, there is provided use of a compound according to any one of (1) to (47) or a pharmacologically acceptable salt or prodrug thereof in the preparation of a medicament for the prophylaxis or treatment of Metabolic Disorders.

In a twenty-first aspect of the present invention, there is provided use of a compound according to any one of (1) to (47) or a pharmacologically acceptable salt or prodrug thereof in the preparation of a medicament for the prophylaxis or treatment of Memory Loss.

In a twenty-second aspect of the present invention, there is provided use of a compound according to any one of (1) to (47) or a pharmacologically acceptable salt or prodrug thereof in the preparation of a medicament for the prophylaxis or treatment of CNS-Mediated Motor Dysfunction Disorders.

In a twenty-third aspect of the present invention, there is provided use of a compound according to any one of (1) to (47) or a pharmacologically acceptable salt or prodrug thereof in the preparation of a medicament for the prophylaxis or treatment of Ophthalmic Disorders.

In an twenty-fourth aspect of the present invention, there is provided a method for the prophylaxis or treatment of a disease in which Kv1.x is involved comprising administering to a patient in need thereof an effective amount of a compound according to any one of (1) to (47) or a pharmacologically acceptable salt or prodrug thereof.

In a twenty-fifth aspect of the present invention, there is provided a method for the prophylaxis or treatment of a condition or disease ameliorated by Kv1.x channel opening comprising administering to a patient in need thereof an effective amount of a compound according to any one of (1) to (47) or a pharmacologically acceptable salt or prodrug thereof.

In a twenty-sixth aspect of the present invention, there is provided a method for the prophylaxis or treatment of a condition or disease ameliorated by Kv1.x channel inhibition comprising administering to a patient in need thereof an effective amount of a compound according to any one of (1) to (47) or a pharmacologically acceptable salt or prodrug thereof.

In a twenty-seventh aspect of the present invention, there is provided a method for the prophylaxis or treatment of Lower Urinary Tract Disorders comprising administering to a patient in need thereof an effective amount of a compound according to any one of (1) to (47) or a pharmacologically acceptable salt or prodrug thereof.

In a twenty-eighth aspect of the present invention, there is provided a method for the prophylaxis or treatment of Anxiety and Anxiety-Related Conditions comprising administering to a patient in need thereof an effective amount of a compound according to any one of (1) to (47) or a pharmacologically acceptable salt or prodrug thereof.

In a twenty-ninth aspect of the present invention, there is provided a method for the prophylaxis or treatment of Epilepsy comprising administering to a patient in need thereof an effective amount of a compound according to any one of (1) to (47) or a pharmacologically acceptable salt or prodrug thereof.

In a thirtieth aspect of the present invention, there is provided a method for the prophylaxis or treatment of Pain Disorders comprising administering to a patient in need thereof an effective amount of a compound according to any one of (1) to (47) or a pharmacologically acceptable salt or prodrug thereof.

In a thirty-first aspect of the present invention, there is provided a method for the prophylaxis or treatment of Gynaecological Pain comprising administering to a patient in need thereof an effective amount of a compound according to any one of (1) to (47) or a pharmacologically acceptable salt or prodrug thereof.

In a thirty-second aspect of the present invention, there is provided a method for the prophylaxis or treatment of Cardiac Arrhythmias comprising administering to a patient in need thereof an effective amount of a compound according to any one of (1) to (47) or a pharmacologically acceptable salt or prodrug thereof.

In a thirty-third aspect of the present invention, there is provided a method for the prophylaxis or treatment of Thromboembolic Events comprising administering to a patient in need thereof an effective amount of a compound according to any one of (1) to (47) or a pharmacologically acceptable salt or prodrug thereof.

In a thirty-fourth aspect of the present invention, there is provided a method for the prophylaxis or treatment of Cardiovascular Diseases comprising administering to a patient in need thereof an effective amount of a compound according to any one of (1) to (47) or a pharmacologically acceptable salt or prodrug thereof.

In a thirty-fifth aspect of the present invention, there is provided a method for the prophylaxis or treatment of Disorders of the Auditory System comprising administering to a patient in need thereof an effective amount of a compound according to any one of (1) to (47) or a pharmacologically acceptable salt or prodrug thereof.

In a thirty-sixth aspect of the present invention, there is provided a method for the prophylaxis or treatment of Migraine comprising administering to a patient in need thereof an effective amount of a compound according to any one of (1) to (47) or a pharmacologically acceptable salt or prodrug thereof.

In a thirty-seventh aspect of the present invention, there is provided a method for the prophylaxis or treatment of Gastrointestinal Disorders comprising administering to a patient in need thereof an effective amount of a compound according to any one of (1) to (47) or a pharmacologically acceptable salt or prodrug thereof.

In a thirty-eighth aspect of the present invention, there is provided a method for the prophylaxis or treatment of Vascular and Visceral Smooth Muscle Disorders comprising administering to a patient in need thereof an effective amount of a compound according to any one of (1) to (47) or a pharmacologically acceptable salt or prodrug thereof.

In a thirty-ninth aspect of the present invention, there is provided a method for the prophylaxis or treatment of Cell Proliferative Disorders comprising administering to a patient in need thereof an effective amount of a compound according to any one of (1) to (47) or a pharmacologically acceptable salt or prodrug thereof.

In a fortieth aspect of the present invention, there is provided a method for the prophylaxis or treatment of Metabolic Disorders comprising administering to a patient in need thereof an effective amount of a compound according to any one of (1) to (47) or a pharmacologically acceptable salt or prodrug thereof.

In a forty-first aspect of the present invention, there is provided a method for the prophylaxis or treatment of Memory Loss comprising administering to a patient in need thereof an effective amount of a compound according to any one of (1) to (47) or a pharmacologically acceptable salt or prodrug thereof.

In a forty-second aspect of the present invention, there is provided a method for the prophylaxis or treatment of CNS-Mediated Motor Dysfunction Disorders comprising administering to a patient in need thereof an effective amount of a compound according to any one of (1) to (47) or a pharmacologically acceptable salt or prodrug thereof.

In a forty-third aspect of the present invention, there is provided a method for the prophylaxis or treatment of Ophthalmic Disorders comprising administering to a patient in need thereof an effective amount of a compound according to any one of (1) to (47) or a pharmacologically acceptable salt or prodrug thereof.

In a forty-fourth aspect of the present invention there is provided a pharmaceutical composition comprising a pharmacologically acceptable diluent or carrier and at least two active ingredients, wherein said active ingredients comprise at least one compound according to any one of (1) to (47) or a pharmacologically acceptable salt or prodrug thereof in combination with at least one compound selected from the group consisting of muscarinic receptor antagonists, β3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists, alpha-1 adrenoceptor antagonists, tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs).

### DETAILED DESCRIPTION OF THE INVENTION

In the compounds of the present invention, the alkyl groups in the definitions of R1, R2, R3, R4, R5a, R5b, R5c, R6, R7 and R8 are preferably alkyl groups having from 1 to 6 carbon atoms, more preferably alkyl groups having from 1 to 4 carbon atoms and most preferably methyl groups.

In the compounds of the present invention, the cycloalkyl groups in the definition of R1 is preferably a cycloalkyl group having from 3 to 8 carbon atoms, more preferably having from 5 to 7 carbon atoms and most preferably cyclohexyl.

In the compounds of the present invention, the aryl groups in the definitions of R1, R5a, R5b, R5c, R6, R7 and R8 are preferably aryl groups having from one to 5 to 14 carbon atoms which may optionally substituted with at least one substituent selected from alkyl groups, halogen atoms, haloalkyl groups, phenyl groups, alkoxy groups, nitro groups, amino groups, monoalkylamino groups, dialkylamino groups, acylamino groups, alkoxycarbonylamino groups, alkylsulphonyl groups, arylsulphonyl groups, aminosulphonyl groups, alkoxycarbonyl groups, carboxyl groups, hydroxyl groups and cyano groups. Examples of the unsubstituted aryl groups include phenyl, indenyl, naphthyl, phenanthrenyl and anthracenyl groups. More preferred aryl groups include phenyl groups which may optionally substituted by 1 or 2 alkyl groups.

In the compounds of the present invention, the aralkyl groups in the definitions of R1, R5a, R5b, R5c, R6, R7 and R8 are preferably alkyl groups as defined above which are substituted with one or more aryl groups as defined above, and are more preferably benzyl and phenethyl groups.

In the compounds of the present invention, the heteroaryl groups in the definitions of R5a, R5b, R5c, R6 and R8 are preferably 5- to 7-membered aromatic heterocyclic group containing 1 to 3 sulfur atoms, oxygen atoms and/or nitrogen atoms. Examples include furyl, thienyl, pyrrolyl, azepinyl, pyrazolyl, imidazolyl, oxazolyl, isoxazolyl, thiazolyl, isothiazolyl, 1,2,3-oxadiazolyl, triazolyl, tetrazolyl, thiadiazolyl, pyranyl, pyridyl, pyridazinyl, pyrimidinyl and pyrazinyl groups.

In the compounds of the present invention, the heteroaralkyl groups in the definitions of R5a, R5b, R5c, R6 and R8 are preferably alkyl groups as defined above which are substituted with heteroaryl groups as defined above.

In the compounds of the present invention, the alkoxyalkyl group in the definition of R8 is preferably an alkyl group having as defined above which is substituted by an alkoxy group as defined below, and is more preferably an alkyl group having from 1 to 4 carbon atoms which is substituted with an alkoxy group having from 1 to 4 carbon atoms.

In the compounds of the present invention, the haloalkyl groups in the definitions of R2, R3 and R4 are preferably aryl groups having from one to 5 to 14 carbon atoms which may optionally substituted with at least one substituent selected from hydrogen atoms, alkyl groups, halogen atoms, haloalkyl groups, alkoxy groups, alkoxycarbonyl groups, carboxyl groups, hydroxyl groups and cyano groups.

In the compounds of the present invention, the alkoxy groups in the definitions of R2, R3 and R4 are preferably alkoxy groups having from 1 to 6 carbon atoms, more preferably alkoxy groups having from 1 to 4 carbon atoms and most preferably methoxy or ethoxy groups.

In the compounds of the present invention, the alkoxycarbonyl groups in the definitions of R2, R3 and R4 are preferably carbonyl groups substituted with alkoxy groups as defined, and are more preferably methoxycarbonyl or ethoxycarbonyl groups.

In the compounds of the present invention, the monoalkylamino groups in the definitions of R1, R2, R3, R4, R5a, R5b, R5c, R6, R7 and R8 are preferably amino groups which are substituted with one alkyl group as defined above, and are more preferably methylimino, ethylamino or t-butylamino groups.

In the compounds of the present invention, the dialkylamino groups in the definitions of R1, R2, R3, R4, R5a, R5b, R5c, R6, R7 and R8 are preferably amino groups which are substituted with two alkyl groups as defined above which may be the same or different from each other, and are more preferably dimethylamino or diethylamino groups.

In the compounds of the present invention, the acylamino groups in the definitions of R1, R2, R3, R4, R5a, R5b, R5c, R6, R7 and R8 are preferably amino groups which are substituted with an acyl group having from 1 to 6 carbon atoms and are more preferably acetylamino or propanoylamino groups.

In the compounds of the present invention, the alkoxycarbonylamino groups in the definitions of R1, R2, R3, R4, R5a, R5b, R5c, R6, R7 and R8 are preferably amino groups which are substituted with an alkoxycarbonyl group as defined above, and are more preferably methoxycarbonylamino or ethoxycarbonylamino groups.

In the compounds of the present invention, the alkylsulphonyl groups in the definitions of R1, R2, R3, R4, R5a, R5b, R5c, R6, R7 and R8 are preferably sulphonyl groups which are substituted with an alkyl group as defined above and are more preferably a methylsulphonyl or ethylsulphonyl group.

In the compounds of the present invention, the arylsulphonyl groups in the definitions of R1, R2, R3, R4, R5a, R5b, R5c, R6, R7 and R8 are preferably sulphonyl groups which are substituted with an aryl group as defined above and are more preferably a phenylsulphonyl group which may be optionally substituted with one or two alkyl groups as defined above or a naphthylsulphonyl group.

The compounds of formula (1a), 1(b), (1c) or (1d) of the present invention can form pharmacologically acceptable salts and these form a part of the present invention. Examples of such salts include inorganic salts such as ammonium salts; organic amine salts such as t-octylamine salts, dibenzylamine salts, morpholine salts, glucosamine salts, phenylglycine alkyl ester salts, ethylenediamine salts, N-methylglucamine salts, guanidine salts, diethylamine salts, triethylamine salts, dicyclohexylamine salts, N,N′-dibenzylethylenediamine salts, chloroprocaine salts, procaine salts, diethanolamine salts, N-benzyl-N-phenethylamine salts, piperazine salts, tetramethylammonium salts and tris(hydroxymethyl)aminemethane salts; hydrohalogenated salts such as hydrofluoric acid salts, hydrochlorides, hydrobromides and hydroiodides; inorganic acid salts such as nitrates, perchlorates, sulfates and phosphates; lower alkanesulfonate salts such as methanesulfonates, trifluoromethanesulfonates and ethanesulfonates; arylsulfonate salts such as benzensulfonates and p-toluenesulfonates; organic acid salts such as acetates, malates, fumarates, succinates, citrates, tartrates, oxalates and maleates; and amino acid salts such as ornithinates, glutamates and aspartates. Of these, organic amine salts are more preferred and triethylamine salts are most preferred.

The compounds of formula (1a), 1(b), (1c) or (1d) of the present invention can be administered in the form of prodrugs. Prodrugs are derivatives of the pharmacologically active compound in which one or more of the substituents on said compound are protected by a group which is then removable by a biological process (e.g. hydrolysis) in vivo after administration to the patient. Many suitable prodrugs would be well-known to the person in the art and can be found, for example, in “Greene's Protective Groups in Organic Synthesis”, 4^{th} Edition, 2006, Wiley-VCH. Suitable examples of such prodrugs include pharmacologically acceptable esters of the compound having the formula (1a), 1(b), (1c) or (1d) wherein a carboxyl moiety of the compound having the formula (1a), 1(b), (1c) or (1d) is esterified. The pharmacologically acceptable esters are not particularly restricted, and can be selected by a person with an ordinary skill in the art. In the case of said esters, it is preferable that such esters can be cleaved by a biological process such as hydrolysis in vivo. The group constituting the said esters (the group shown as R when the esters thereof are expressed as —COOR) can be, for example, a C₁-C₄ alkoxy C₁-C₄ alkyl group such as methoxyethyl, 1-ethoxyethyl, 1-methyl-1-methoxyethyl, 1-(isopropoxy)ethyl, 2-methoxyethyl, 2-ethoxyethyl, 1,1-dimethyl-1-methoxymethyl, ethoxymethyl, propoxymethyl, isopropoxymethyl, butoxymethyl or t-butoxymethyl; a C₁-C₄ alkoxylated C₁-C₄ alkoxy C₁-C₄ alkyl group such as 2-methoxyethoxymethyl, a C₆-C₁₀ aryloxy C₁-C₄ alkyl group such as phenoxymethyl; a halogenated C₁-C₄ alkoxy C₁-C₄ alkyl group such as 2,2,2-trichloroethoxymethyl or bis(2-chloroethoxy)methyl; a C₁-C₄ alkoxycarbonyl C₁-C₄ alkyl group such as methoxycarbonylmethyl; a cyano C₁-C₄ alkyl group such as cyanomethyl or 2-cyanoethyl; a C₁-C₄ alkylthiomethyl group such as methylthiomethyl or ethylthiomethyl; a C₆-C₁₀ arylthiomethyl group such as phenylthiomethyl or naphthylthiomethyl; a C₁-C₄ alkylsulfonyl C₁-C₄ lower alkyl group, which may be optionally substituted with a halogen atom(s) such as 2-methanesulfonylethyl or 2-trifluoromethanesulfonylethyl; a C₆-C₁₀ arylsulfonyl C₁-C₄ alkyl group such as 2-benzenesulfonylethyl or 2-toluenesulfonylethyl; a C₁-C₇ aliphatic acyloxy C₁-C₄ alkyl group such as formyloxymethyl, acetoxymethyl, propionyloxymethyl, butyryloxymethyl, pivaloyloxymethyl, valeryloxymethyl, isovaleryloxymethyl, hexanoyloxymethyl, 1-formyloxyethyl, 1-acetoxyethyl, 1-propionyloxyethyl, 1-butyryloxyethyl, 1-pivaloyloxyethyl, 1-valeryloxyethyl, 1-isovaleryloxyethyl, 1-hexanoyloxyethyl, 2-formyloxyethyl, 2-acetoxyethyl, 2-propionyloxyethyl, 2-butyryloxyethyl, 2-pivaloyloxyethyl, 2-valeryloxyethyl, 2-isovaleryloxyethyl, 2-hexanoyloxyethyl, 1-formyloxypropyl, 1-acetoxypropyl, 1-propionyloxypropyl, 1-butyryloxypropyl, 1-pivaloyloxypropyl, 1-valeryloxypropyl, 1-isovaleryloxypropyl, 1-hexanoyloxypropyl, 1-acetoxybutyl, 1-propionyloxybutyl, 1-butyryloxybutyl, 1-pivaloyloxybutyl, 1-acetoxypentyl, 1-propionyloxypentyl, 1-butyryloxypentyl, 1-pivaloyloxypentyl or 1-pivaloyloxyhexyl; a C₅-C₆ cycloalkylcarbonyloxy C₁-C₄ alkyl group such as cyclopentylcarbonyloxymethyl, cyclohexylcarbonyloxymethyl, 1-cyclopentylcarbonyloxyethyl, 1-cyclohexylcarbonyloxyethyl, 1-cyclopentylcarbonyloxypropyl, 1-cyclohexylcarbonyloxypropyl, 1-cyclopentylcarbonyloxybutyl or 1-cyclohexylcarbonyloxybutyl; a C₆-C₁₀ arylcarbonyloxy C₁-C₄ alkyl group such as benzyloxymethyl; a C₁-C₆ alkoxycarbonyloxy C₁-C₄ alkyl group such as methoxycarbonyloxymethyl, 1-(methoxycarbonyloxy)ethyl, 1-(methoxycarbonyloxy)propyl, 1-(methoxycarbonyloxy)butyl, 1-(methoxycarbonyloxy)pentyl, 1-(methoxycarbonyloxy)hexyl, ethoxycarbonyloxymethyl, 1-(ethoxycarbonyloxy)ethyl, 1-(ethoxycarbonyloxy)propyl, 1-(ethoxycarbonyloxy)butyl, 1-(ethoxycarbonyloxy)pentyl, 1-(ethoxycarbonyloxy)hexyl, propoxycarbonyloxymethyl, 1-(propoxycarbonyloxy)ethyl, 1-(propoxycarbonyloxy)propyl, 1-(propoxycarbonyloxy)butyl, isopropoxycarbonyloxymethyl, 1-(isopropoxycarbonyloxy)ethyl, 1-(isopropoxycarbonyloxy)butyl, butoxycarbonyloxymethyl, 1-(butoxycarbonyloxy)ethyl, 1-(butoxycarbonyloxy)propyl, 1-(butoxycarbonyloxy)butyl, isobutoxycarbonyloxymethyl, 1-(isobutoxycarbonyloxy)ethyl, 1-(isobutoxycarbonyloxy)propyl, 1-(isobutoxycarbonyloxy)butyl, t-butoxycarbonyloxymethyl, 1-(t-butoxycarbonyloxy)ethyl, pentyloxycarbonyloxymethyl, 1-(pentyloxycarbonyloxy)ethyl, 1-(pentyloxycarbonyloxy)propyl, hexyloxycarbonyloxymethyl, 1-(hexyloxycarbonyloxy)ethyl or 1-(hexyloxycarbonyloxy)propyl; a C₅-C₆ cycloalkyloxycarbonyloxy C₁-C₄ alkyl group such as cyclopentyloxycarbonyloxymethyl, 1-(cyclopentyloxycarbonyloxy)ethyl, 1-(cyclopentyloxycarbonyloxy)propyl, 1-(cyclopentyloxycarbonyloxy)butyl, cyclohexyloxycarbonyloxymethyl, 1-(cyclohexyloxycarbonyloxy)ethyl, 1-(cyclohexyloxycarbonyloxy)propyl or 1-(cyclohexyloxycarbonyloxy)butyl; a [5-(C₁-C₄ alkyl)-2-oxo-1,3-dioxolen-4-yl]methyl group such as (5-methyl-2-oxo-1,3-dioxolen-4-yl)methyl, (5-ethyl-2-oxo-1,3-dioxolen-4-yl)methyl, (5-propyl-2-oxo-1,3-dioxolen-4-yl)methyl, (5-isopropyl-2-oxo-1,3-dioxolen-4-yl)methyl or (5-butyl-2-oxo-1,3-dioxolen-4-yl)methyl; a [5-(phenyl, which may be optionally substituted with a C₁-C₄ alkyl, C₁-C₄ alkoxy or halogen atom(s))-2-oxo-1,3-dioxolen-4-yl]methyl group such as (5-phenyl-2-oxo-1,3-dioxolen-4-yl)methyl, [5-(4-methylphenyl)-2-oxo-1,3-dioxolen-4-yl]methyl, [5-(4-methoxyphenyl)-2-oxo-1,3-dioxolen-4-yl]methyl, [5-(4-fluorophenyl)-2-oxo-1,3-dioxolen-4-yl]methyl or [5-(4-chlorophenyl)-2-oxo-1,3-dioxolen-4-yl]methyl; or a phthalidyl group, which may be optionally substituted with a C₁-C₄ alkyl or C₁-C₄ alkoxy group(s), such as phthalidyl, dimethylphthalidyl or dimethoxyphthalidyl, and is preferably a pivaloyloxymethyl group, phthalidyl group or (5-methyl-2-oxo-1,3-dioxolen-4-yl)methyl group, and more preferably a (5-methyl-2-oxo-1,3-dioxolen-4-yl)methyl group.

The compounds of formula (1a), 1(b), (1c) or (1d) or pharmacologically active salts and prodrugs thereof contain some substituents for which there exist isosteres, and compounds containing such isosteres in place of said substituents also form a part of the present invention. For example, where the compounds of formula (1a), 1(b), (1c) or (1d) or pharmacologically active salts and prodrugs thereof contain a carboxyl group, this can be replaced with a tetrazolyl group.

Hydrates or solvates of the compounds of formula (1a), 1(b), (1c) or (1d), or pharmacologically acceptable salts and prodrugs thereof can also be used and form a part of the invention.

Some compounds of formula (1a), 1(b), (1c) or (1d) and their pharmacologically acceptable salts and prodrugs thereof of the present invention may have one or more asymmetric carbons, and optical isomers (including diastereomers) due to the presence of asymmetric carbon atom(s) in the molecule can exist. These respective isomers are included in the present invention, both as individual isomers and mixtures thereof in all possible ratios.

Compounds of formula (I) in which X and Y are hydrogen or one of X and Y is H and the other is a suitable protecting group are reacted with electrophiles such as acyl chlorides, anhydrides, sulphonyl chlorides, chloroformates, isoycanates or sulfonylisocyanates to give the compounds of the invention after removal of the protecting group if appropriate.

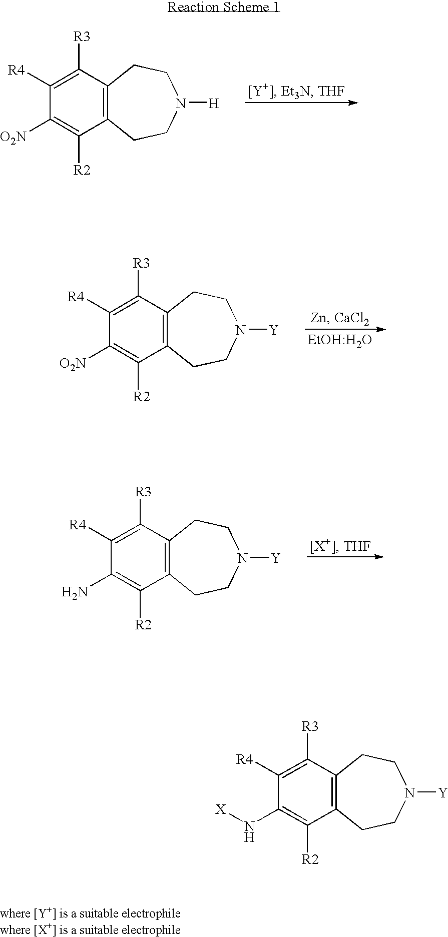

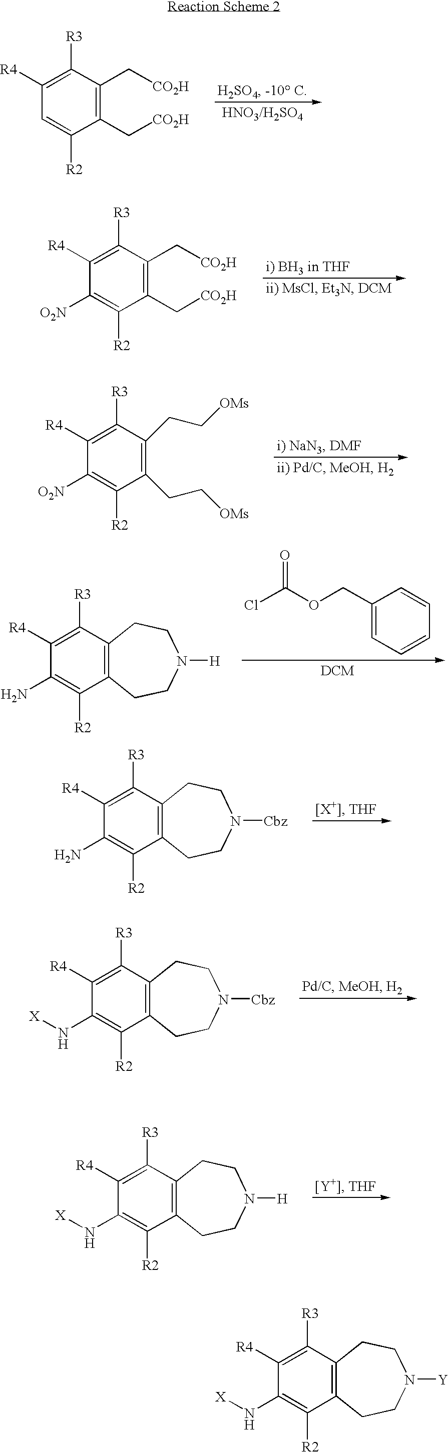

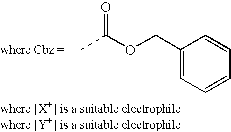

Examples of the administration form of a compound having the general formula (1a), 1(b), (1c) or (1d) of the present invention, or a pharmacologically acceptable salt or prodrug thereof include oral administration by tablets, capsules, granules, powders or syrups, and parenteral administration by injection, pathches or suppositories. Moreover, a compound having the general formula (1a), 1(b), (1c) or (1d) or a pharmacologically acceptable salt or prodrug thereof of the present invention can also be administered by pulmonary administration in the form of a powder, solution or suspension. Preparations for these administrations are produced by known methods using additives such as excipients, lubricants, binders, disintegrants, stabilizers, corrigents, diluents and so forth.

Examples of excipients include organic excipients such as sugar derivatives, e.g. lactose, sucrose, glucose, mannitol or sorbitol, starch derivatives, e.g. corn starch, potato starch, α-starch, dextrin or carboxymethyl starch, cellulose derivatives, e.g. crystalline cellulose, low substituted hydroxypropyl cellulose, hydroxypropyl methyl cellulose, carboxymethyl cellulose, calcium carboxymethyl cellulose or internally crosslinked sodium carboxymethyl cellulose, and gum Arabic, dextran or pullulan; and, inorganic excipients such as silicate derivatives, e.g. light anhydrous silicic acid, synthetic aluminum silicate or magnesium aluminum metasilicate, phosphates, e.g. calcium phosphate, carbonates, e.g. calcium carbonate, or sulfates, e.g. calcium sulfate.

Examples of lubricants include stearic acid and metal stearates such as calcium stearate or magnesium stearate; talc; colloidal silica; waxes such as bee gum or spermaceti; boric acid; adipic acid; sulfates such as sodium sulfate; glycol; fumaric acid; sodium benzoate; (D/L)-leucine; sodium fatty acid salts; lauryl sulfates such as sodium lauryl sulfate or magnesium lauryl sulfate; silicic acids such as silicic anhydride or silicate hydrate; and, starch derivatives.

Examples of binders include polyvinylpyrrolidone, Macrogol and compounds similar to the aforementioned excipients.

Examples of disintegrants agents include compounds similar to the aforementioned excipients, and chemically crosslinked starches and celluloses such as cross sodium carmellose, sodium carboxymethyl starch or crosslinked polyvinylpyrrolidone.

Examples of stabilizers include paraoxybenzoate esters such as methyl paraben or propyl paraben; alcohols such as chlorobutanol, benzyl alcohol or phenyl ethyl alcohol; benzalkonium chloride; phenols such as phenol or cresol; thimerosal; dehydroacetic acid; and, sorbic acid.

Examples of corrigents include ordinarily used sweeteners, sour flavourings and fragrances.

In the case of producing a solution or suspension for pulmonary administration of a compound having the general formula (1a), 1(b), (1c) or (1d) or a pharmacologically acceptable salt or prodrug thereof of the present invention, for example, said solution or suspension can be produced by dissolving or suspending crystals of the present invention in water or in a mixture of water and an auxiliary solvent (e.g. ethanol, propylene glycol or polyethylene glycol). Such a solution or suspension may also contain an antiseptic (e.g. benzalkonium chloride), solubilizing agent (e.g. a polysorbate such as Tween 80 or Span 80 or surface activator such as benzalkonium chloride), buffer, isotonic agent (e.g. sodium chloride), absorption promoter and/or thickener. In addition, the suspension may additionally contain a suspending agent (such as microcrystalline cellulose or sodium carboxymethyl cellulose).

A composition for pulmonary administration produced in the manner described above is administered directly into the nasal cavity or oral cavity by a typical means in the field of inhalants (using, for example, a dropper, pipette, cannula or atomizer). In the case of using an atomizer, crystals of the present invention can be atomized as an aerosol in the form of a pressurized pack together with a suitable nebula (for example, a chlorofluorocarbon such as dichlorofluoromethane, trichlorofluoromethane or dichlorotetrafluoroethane, or a gas such as carbon dioxide), or they can be administered using a nebulizer.

The amount of a compound having the general formula (1a), 1(b), (1c) or (1d) or pharmacologically acceptable salt or prodrug thereof of the present invention used varies depending on the symptoms, age, administration method and so forth, and may be administered either in a single dose or by dividing into multiple doses according to the symptoms.

In the combinations according to the forty-seventh aspect of the present invention, typical examples of each of the classes of compounds that can be used in combination with the compounds having the general formula (1a), (1b), 1c) or (1d) or a pharmacologically acceptable salt or prodrug thereof of the present invention are as follows:

1. Examples of muscarinic receptor antagonists (including but not limited to selective M3 antagonists) include esoxybutynin, oxybutynin [especially the chloride], tolterodine [especially the tartrate], solifenacin [especially the succinate], darifenacin [especially the hydrobromide], temiverine, fesoterodine, imidafenacin and trospium [especially the chloride].
2. Examples of β3 adrenergic receptor agonists include YM-178 and solabegron, KUC-7483.
3. Examples of neurokinin K receptor antagonists (including selective NK-1 antagonists) include cizolirtine and casopitant.
4. Examples of vanilloid VR1 agonists include capsaicin, resiniferatoxin and NDG-8243.
5. Examples of calcium channel α2 δ ligands include gabapentin and pregabalin.
6. Examples of potassium channel activators (including activators of KCNQ, BKCa channels, Kv channels and KATP channels) include KW-7158, NS-8 and retigabine.
7. Examples of calcium channel inhibitors (including Cav2.2 channel inhibitors) include ziconotide and NMED-160.
8. Examples of sodium channel blockers include lidocaine, lamotrigine, VX-409, ralfinamide and carbamazepine.
9. Examples of serotonin and norepinephrine reuptake inhibitors (SNRIs) include duloxetine and venlafaxine.
10. Examples of 5-HT antagonists including 5-HT1a antagonists and 5HT3 antagonists.
11. Examples of α-1 adrenoceptor antagonists include tamsulosin.
12. Examples of tricyclic antidepressants include amitriptyline, amoxapine, clomipramine, dosulepin (dothiepin), doxepin, imipramine, lofepramine, nortriptyline, and trimipramine.
13. Examples of N-methyl-D-aspartate (NMDA) receptor antagonists include ketamine, memantine, amantadine, AVP-923, NP-1 and EVT-101.
14. Examples of cannabinoid receptor agonists include GW-1000 (Sativex) and KDS-2000.
15. Anti-convulsants. Examples include lacosamide, carbamazepine, topiramate, oxcarbazepine and levetiracetam.
16. Examples of aldose reductase inhibitors include tolrestat, zopolrestat, zenarestat, epalrestat, sorbinil, AS-3201, fidarestat, risarestat, ponalrestat and alrestatin.
17. Examples of opioids (e.g. mu opioid agonists) include fentanyl and tapentadol.
18. Examples of alpha adrenoceptor agonists include a₁-adrenoceptor agonists such as ethoxyamine, phenylephrine, oxymetazoline, tetrahydralazine and xylometazoline and a₂-adrenoceptor agonists such as clonidine, guanabenz, guanfacine and α-methyldopa.
19. Examples of P2X receptor antagonists including P2X2 receptor antagonists and P2X7 receptor antagonists.
20. Examples of acid-sensing ion channel modulators include amiloride.
21. Examples of NGF receptor modulators include trkA.
22. Examples of nicotinic acetylcholine receptor modulators include A-85380, tebanicline, ABT-366833, ABT-202, ABT-894, epibatidine analogs and SIB-1663.
23. Examples of synaptic vesicle protein 2A ligands include brivaracetam.

Examples of the administration form of the combination of the present invention are the same as given above for the compounds of general formula (1a), (1b), (1c) and (1d) and pharmacologically acceptable salts and prodrugs thereof. The particular form can be chosen depending upon the condition to be treated and the nature of the compounds being administered in combination. For example, a combination of a compound of general formula (1a), (1b), (1c) and (1d) or a pharmacologically acceptable salt thereof with lidocaine could be administered transdermally by means of a patch while a combination with ziconotide could be administered transmucosally.

### Synthesis of the Compounds of the Invention

### EXAMPLES

### Examples 1 to 5

7-Nitro-1,2,4,5-tetrahydro-3H-3-benzazepine was prepared as described in EP284384.

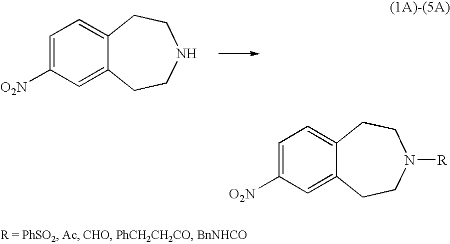

To a solution of 7-Nitro-1,2,4,5-tetrahydro-3H-3-benzazepine (1.0 equiv.) in tetrahydrofuran (approx. 0.25M) under an atmosphere of nitrogen, was added an electrophile from Table 1 below (1.1 equiv.). Triethylamine (1.1 equiv.) was added as indicated in Table 1 below. The reaction mixture was then stirred overnight before dilution with ethyl acetate. The organic phase was then washed with water dried over magnesium sulfate, filtered and the solvent removed under reduced pressure. The crude reaction mixtures were purified by column chromatography (silica, typically 1:1 ethyl acetate: hexane) to give the intermediates (1A)-(5A).

TABLE 1

| Example | Electrophile | Et₃N added? | Yield |
|---|---|---|---|
| 1A | PhSO₂Cl | Yes | 43% |
| 2A | AcCl | Yes | 46% |
| 3A | BnNCO | No | 73% |
| 4A | PhCH₂CH₂COCl | Yes | 74% |
| 5A | AcOCHO | Yes | 56% |

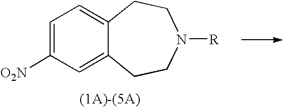

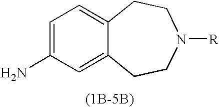

R = PhSO₂, Ac, CHO, PhCH₂CH₂CO, BnNHCO

To a solution of the N-substituted benzazepines of formula (1A)-(5A) prepared above (1.0 equiv.) in EtOH:H₂O (8:2, 0.01M) was added Zn (30 equiv.) and CaCl₂ (0.6 equiv.). The reaction mixture was heated to reflux for 2 hours until TLC analysis indicated consumption of starting material. The reaction mixture was filtered whilst hot, cooled and the solvent removed under reduced pressure. The resultant crude reaction mixtures were purified by column chromatography (silica, typically 2:1 ethyl acetate:hexane) to give the intermediates (1B)-(5B) in the yields given in Table 2 below.

TABLE 2

| Example | R | Yield |
|---|---|---|
| 1B | PhSO₂ | 99% |
| 2B | Ac | 78% |
| 3B | BnNHCO | 93% |
| 4B | PhCH₂CH₂CO | 93% |
| 5B | CHO | 74% |

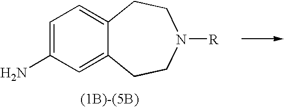

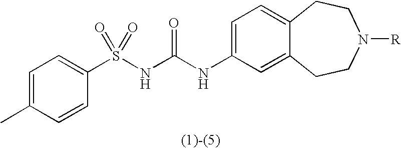

R = PhSO₂, Ac, CHO, PhCH₂CH₂CO, BnNHCO

To a solution of the N-substituted benzazepines of formula (1B)-(5B) (1.0 equiv.) in THF (approx. 0.25M) under an atmosphere nitrogen, was added toluenesulphonylisocyanate (1.0 equiv.). The reaction mixture was stirred overnight before removal of the solvent under reduced pressure. The crude reaction mixture was purified by column chromatography (silica, typically 1:7:92 triethylamine:methanol:chloroform) to furnish the triethylamine salt of the product. This was dissolved in chloroform and washed with 1M citric acid, dried over magnesium sulfate, filtered and the solvent removed under reduced pressure to furnish the desired final compounds of formula (1a), (1b), (1c) or (1d) in the yields given in Table 3 below.

TABLE 3

| Example No. | R | Yield |
|---|---|---|
| 1 | PhSO₂ | 99% |
| 2 | Ac | 42% |
| 3 | BnNHCO | 87% |
| 4 | PhCH₂CH₂CO | 49% |
| 5 | CHO | 56% |

Example 1 N-phenylsulfonyl-7-[({[(4-methylphenyl)sulfonyl]amino}carbonyl)amino]-1,2,4,5-tetrahydro-3H-3-benzazepine-3-carboxamide m/z=500 (ES+, M+H)

Example 2 N-acetyl-7-[({[(4-methylphenyl)sulfonyl]amino}carbonyl)amino]-1,2,4,5-tetrahydro-3H-3-benzazepine-3-carboxamide m/z=402 (ES+, M+H)

Example 3 N-benzylaminocarbonyl-7-[({[(4-methylphenyl)sulfonyl]amino}carbonyl)amino]-1,2,4,5-tetrahydro-3H-3-benzazepine-3-carboxamide m/z=493 (ES+, M+H)

Example 4 N-(1-oxo-3-phenyl-propanyl)-7-[({[(4-methylphenyl)sulfonyl]amino}carbonyl)amino]-1,2,4,5-tetrahydro-3H-3-benzazepine-3-carboxamide m/z=492 (ES+, M+H)

Example 5 N-formyl-7-[({[(4-methylphenyl)sulfonyl]amino}carbonyl)amino]-1,2,4,5-tetrahydro-3H-3-benzazepine-3-carboxamide m/z=410 (ES+, MNa⁺)

### Examples 6 to 10

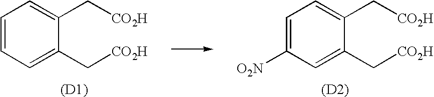

To a solution of the (2-carboxymethyl-phenyl)acetic acid (D) (25.0 g, 0.13 mol) in H₂SO₄ (85 ml) at −10° C. was added HNO₃ (10 ml) in H₂SO₄ (4 ml). The reaction mixture was stirred for 4 hours at −10° C. before warming to room temperature. The reaction mixture was then poured onto ice (500 ml) and extracted with ethyl acetate. The organic phase was dried over magnesium sulfate, filtered and the solvent removed under reduced pressure. The crude reaction mixture was purified by recrystallisation from ethyl acetate/hexanes to furnish the desired intermediate (D2) as a colourless solid (21.38 g, 69%).

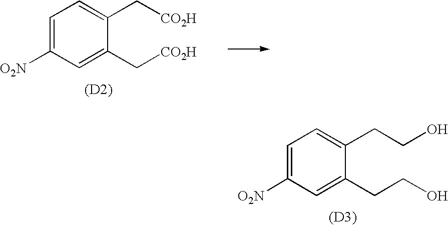

To a solution of the intermediate (D2) (10.9 g, 0.05 mol) in THF (180 ml) under an atmosphere of nitrogen at 0° C. was added borane (100 ml, 1.0M in THF) over 1 hour. The reaction mixture was then stirred for 1 hour at 0° C. before warming to room temperature and stirring for a further 4 hours. The reaction mixture was quenched with water (100 ml) and extracted with ethyl acetate. The combined organic phases were washed with 1M NaOH, dried over magnesium sulfate, filtered and the solvent removed under reduced pressure. The desired diol intermediate (D3) was isolated as an orange oil (8.01 g, 83%) that was used without further purification.

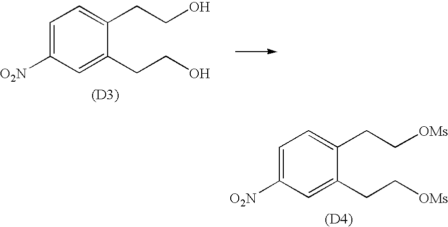

To a solution of the diol intermediate (D3) (7.95 g, 35.6 mmol) in DCM (200 ml) was added Et₃N (29.7 ml, 213 mmol). The reaction mixture was cooled to 0° C. and methanesulphonyl chloride (10.2 ml, 124 mmol) added. The reaction mixture was stirred, with slow warming to room temperature, overnight before dilution with water and extraction with DCM. The organic phases were dried over magnesium sulfate, filtered and the solvent removed under reduced pressure. The crude reaction mixture was redissolved in the minimum volume of DCM and precipitated with hexanes. The resultant intermediate product (D4) was isolated by filtration as a colourless solid (11.0 g, 85%).

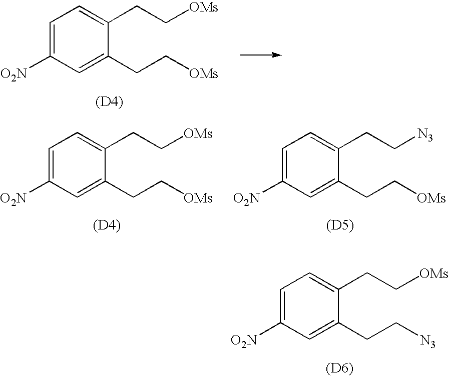

To a solution of the dimesylate (D4) (4.7 g, 12.8 mmol) prepared above in DMF (25 ml) under an atmosphere of nitrogen was added NaN₃ (498 mg, 7.66 mmol) in two portions over two hours. The reaction mixture was stirred for a further two hours before dilution with ethyl acetate and washing of the organic phases with water. The organic phase was dried over magnesium sulfate, filtered and the solvent removed under reduced pressure. The crude reaction mixture was purified by column chromatography (silica, 1:1 ethyl acetate:hexane to 2:1 ethyl acetate:hexane to 4:1 ethyl acetate:hexane) to furnish a mixture of monoazides (D5) and (D6) (1.24 g, 31%) and dimesylate (2.8 g, 60%). The recovered dimesylate (D4) was resubmitted to the above reaction conditions to give an overall yield of monoazides (D5) and (D6) (2.23 g, 55%).

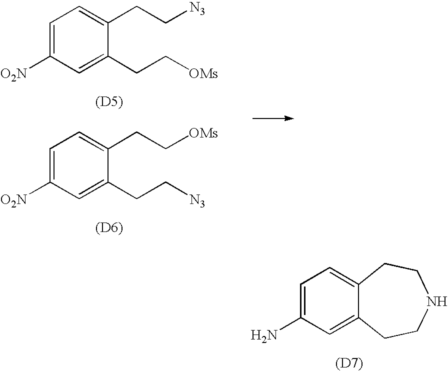

To a mixture of the monoazides (D5) and (D6) prepared above (1.75 g, 5.56 mmol) in degassed MeOH (150 ml) was added Pd/C (300 mg, 20% w/w). The reaction mixture was stirred under an atmosphere of hydrogen for 4 hours before filtration through a pad of celite and the solvent removed under reduced pressure. The crude product was then passed through a short pad of basic alumina and washed though with MeOH. Removal of the solvent furnished 7-amino-1,2,4,5-tetrahydro-3H-3-benzazepine (D7) as a pale yellow solid (568 mg, 63%).

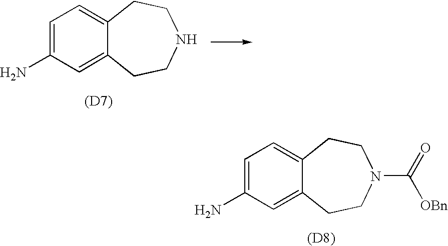

Benzyl chloroformate (13 μl, 92.5 μmol) was added drop wise in DCM (10 ml) to a solution of 7-amino-1,2,4,5-tetrahydro-3H-3-benzazepine (D7) prepared above (15 mg, 92.5 μmol) in DCM (10 ml) at 0° C. over 45 mins. Once addition was complete, stirring was continued for a further 4 hours after which time TLC analysis indicated the formation of a single new product. The reaction mixture was washed with water, dried over magnesium sulfate and filtered. Removal of solvent under reduced pressure furnished benzyl 7-amino-1,2,4,5-tetrahydro-3H-3-benzazepine-3-carboxylate (D8) as a colourless oil (26 mg, 96%) that was used without further purification.

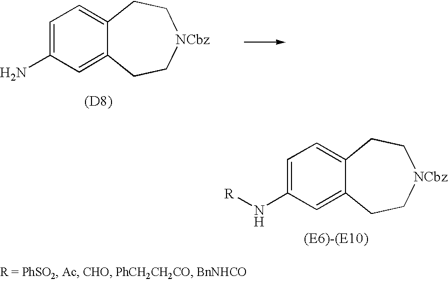

To a solution of benzyl 7-amino-1,2,4,5-tetrahydro-3H-3-benzazepine-3-carboxylate (D8) prepared above (1.0 equiv.) in THF (approx. 0.25M) under an atmosphere of nitrogen, was added the electrophile given in Table 4 below (1.1 equiv.). Et₃N (1.1 equiv.) was added as indicated below. The reaction mixture was then stirred overnight before dilution with ethyl acetate. The organic phase was then washed with water before drying over magnesium sulfate, filtering and concentration under reduced pressure. If necessary, the crude reaction mixtures were purified by column chromatography (silica, typically 1:1 ethyl acetate:hexane) to give the desired intermediates of formula (E6)-(E10).

TABLE 4

| Example | Electrophile | Et₃N added? | Yield |
|---|---|---|---|
| E6 | PhSO₂Cl | Yes | 67% |
| E7 | AcCl | Yes | Quant. |
| | | | (used without |
| | | | purification) |
| E8 | BnNCO | No | 99% |
| | | | (used without |
| | | | purification) |
| E9 | PhCH₂CH₂COCl | Yes | 97% |
| E10 | AcOCHO | Yes | 63% |

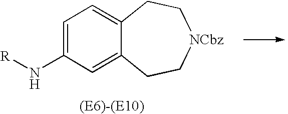

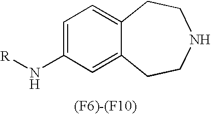

R = PhSO₂, Ac, CHO, PhCH₂CH₂CO, BnNHCO

To a degassed solution of N-substituted benzazepines (E6)-(E10) prepared above in MeOH:THF (2:1 approx 0.08M) was added Pd/C (20% w/w). The reaction mixture was stirred under an atmosphere of hydrogen until TLC analysis showed consumption of starting material. The reaction mixture was filtered through a pad of Celite, washing through with methanol before removal of the solvent under reduced pressure. The desired intermediates of formula (F6)-(F10) were isolated in quantitative yield and were used without further purification.

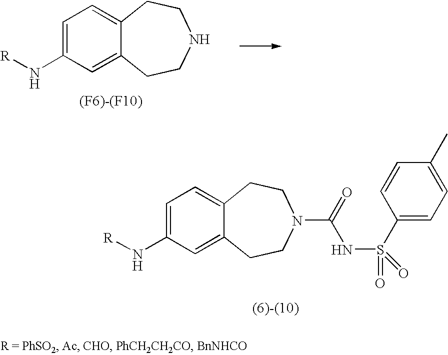

To a solution of the N-substituted benzazepines of formula (F6)-(F10) (1.0 equiv.) in THF (approx. 0.25M) under nitrogen, was added toluenesulphonylisocyanate (1.0 equiv.). The reaction mixture was stirred overnight before removal of the solvent under reduced pressure. The crude reaction mixture was purified by column chromatography (silica, typically 1:7:92 triethylamine:methanol:chloroform) to furnish the triethylamine salt of the product. This was dissolved in chloroform and washed with 1M citric acid, dried over magnesium sulfate, filtered and the solvent removed under reduced pressure to furnish the desired compounds of formula (1a), 1(b), (1c) or (1d) in the yields shown in Table 5 below.

TABLE 5

| Example | Example No. | R | Yield |
|---|---|---|---|
| 6 | 6 | PhSO₂ | 99% |
| 7 | 7 | Ac | 78% |
| 8 | 8 | BnNHCO | 93% |
| 9 | 9 | PhCH₂CH₂CO | 93% |
| 10 | 10 | CHO | 74% |

Example 6 N-[(4-methylphenyl)sulfonyl]-7-phenylsulfonylamino-1,2,4,5-tetrahydro-3H-3-benzazepine-3-carboxamide m/z=522 (ES+, MNa⁺)

Example 7 N-[(4-methylphenyl)sulfonyl]-7-acetylamino-1,2,4,5-tetrahydro-3H-3-benzazepine-3-carboxamide m/z=424 (ES+, MNa⁺)

Example 8 N-[(4-methylphenyl)sulfonyl]-7-benzylaminocarbonylamino-1,2,4,5-tetrahydro-3H-3-benzazepine-3-carboxamide m/z=515 (ES+, M+H)

Example 9 N-[(4-methylphenyl)sulfonyl]-7-(1-oxo-3-phenyl-propanylamino)-1,2,4,5-tetrahydro-3H-3-benzazepine-3-carboxamide m/z=514 (ES+, MNa⁺)

Example 10 N-[(4-methylphenyl)sulfonyl]-7-formylamino-1,2,4,5-tetrahydro-3H-3-benzazepine-3-carboxamide m/z=410 (ES+, MNa⁺)

### Example 11

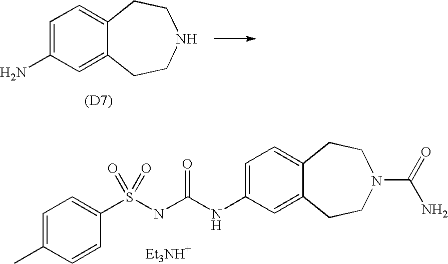

To a solution of 7-amino-1,2,4,5-tetrahydro-3H-3-benzazepine (D7) (51 mg, 0.32 mmol), prepared as described in Examples 6-10 above, in CHCl₃ (3 ml) at 0° C. under an atmosphere of nitrogen was added trimethylsilylisocyanate (43 μl, 0.32 mmol) in CHCl₃ (2 ml) over 10 mins. The reaction mixture was stirred for a further 35 mins before p-toluenesulphonylisocyanate (49 μl, 0.32 mmol) was added. Following stirring for a further 1.5 hours at room temperature a thick precipitate developed. The reaction mixture was then heated to reflux for 30 mins before cooling to room temperature. The solvent was removed under reduced pressure and the crude product dissolved in MeOH (10 ml) and triethylamine (0.5 ml) added. The solvent was then removed under reduced pressure and the resultant crude material purified by column chromatography (silica, gradient elution, 89:10:1 chloroform: methanol: triethylamine to 80:9:1 methanol:chloroform: triethylamine) to furnish 7-[({[(4-methylphenyl)-sulfonyl]amino}carbonyl)amino]-1,2,4,5-tetrahydro-3H-3-benzazepine-3-carboxamide as its triethylamine salt (3 mg) m/z=401 (ES−, M−H).

### Example 12

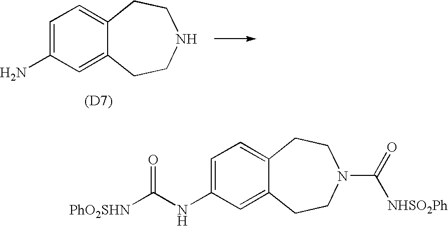

To a solution of 7-amino-1,2,4,5-tetrahydro-3H-3-benzazepine (D7) (21 mg, 0.13 mmol), prepared as described in Examples 6-10 above, in THF (5 ml) was added benzenesulfonylisocyanate (35 μl, 0.26 mmol) under an atmosphere of nitrogen. The reaction mixture was stirred overnight before removal of solvent under reduced pressure. The crude reaction mixture was purified by column chromatography (silica, 1:9:90 triethylamine:methanol:chloroform) to furnish the triethylamine salt of the product. This was dissolved in chloroform and washed with 1M citric acid, dried over magnesium sulfate, filtered and the solvent removed under reduced pressure to furnish N-phenylsulfonyl-7-phenylsulfonylaminocarbonylamino]-1,2,4,5-tetrahydro-3H-3-benzazepine-3-carboxamide (52 mg, 76%) m/z=551 (ES+, MNa⁺).

### Example 13

To a mixture of 6-amino-1,2,3,4-tetrahydroisoquinoline hydrochloride (100 mg, 0.54 mmol), DMF (0.5 ml) and triethylamine (0.2 ml) was added p-toluenesulphonylisocyanate (223 mg, 1.13 mmol) and the mixture stirred at room temperature overnight. The mixture was poured onto water (20 ml) and acidified with 0.5M HCl(aq) to pH 1. The solid was quickly filtered off, washed with water (5 ml) and purified by preparative HPLC. The column fractions were combined and NaCl (s) added to separate the organic solvent from the water. The organic layer was separated and the aqueous layer extracted with MeCN (3 50 ml). The combined organic extracts were evaporated under reduced pressure (water bath below 35° C.) to ca. 10 ml volume then DCM (50 ml) added, the aqueous layer separated and the organic layer dried (Na₂SO₄), filtered and evaporated under reduced pressure (water bath below 35° C.) to give N-[(4-methylphenyl)sulfonyl]-6-({[(4-methylphenyl)sulfonyl]-carbamoyl}amino)-3,4-dihydroisoquinoline-2(1H)-carboxamide as a glass (20 mg, 7%). [M+H]+=543.

### Example 14

To a cooled mixture of sulphuric acid (5.5 ml, 102.8 mmol) (−10° C.) was added isoindoline (2.45 g, 20.6 mmol). The mixture was stirred and kept at 10° C. Nitric acid (2 ml, 41.1 mmol) was added dropwise over 5 mins (temperature increase +10° C.) and stirred for 20 mins at room temperature, then heated at 50° C. for 30 mins. The mixture was then cooled to room temperature and EtOAc (10 mL) was added, the solid was filtered and washed with EtOAc (10 mL). The solid was dried in vacuo to give 5-nitroisoindoline hydrosulfate as a white solid (4.04 g, 75%). mp>240° C. dec. NMR (300 MHz, d6-DMSO) δ 9.63 (1H, br s, NH), 8.28 (1H, s, ArH), 8.21 (1H, dd, ArH), 7.65 (1H, d, ArH), 4.60 (4H, d, 2×CH2).

5-Nitroisoindoline hydrosulfate (1.0 g, 3.81 mmol) was dissolved in hot MeOH (80 ml) and placed in a 300 ml stainless steel autoclave. 5% Pd/C (120 mg) in PhMe (1 ml) was added to the solution and the mixture was charged with hydrogen (15 atms) and left stirring at room temperature for 20 hours. The mixture was filtered through celite, the celite was washed with water (10 ml) to dissolve the product. The solution was concentrated under reduced pressure until a precipitate formed. The mixture was cooled (0° C.), filtered and dried in vacuo to give isoindolin-5-amine hydrosulfate as a white solid (560.7 mg, 63%). mp 240° C. dec. NMR (300 MHz, d6-DMSO) δ 6.98 (1H, d, ArH), 6.52 (1H, d, ArH), 6.49 (1H, s, ArH), 4.29 (4H, d, 2×CH2).

To a suspension of isoindolin-5-amine hydrosulfate (560 mg, 2.4 mmol) in DCM (10 ml) was added triethylamine (0.37 ml, 2.7 mmol) followed by p-toluenesulfonyl isocyanate (0.92 ml, 6.0 mmol). The reaction was stirred at room temperature overnight. The reaction mixture was chromatographed eluting with a gradient of DCM to 10% MeOH:DCM to give the product which was then recolumned eluting with a gradient of DCM to 2.5% MeOH:DCM to give the product. The product was then dissolved in a minimum amount of DCM (with a few drops of MeOH) and Et₂O added resulting in precipitation of a solid which was filtered and dried in vacuo to give N-[(4-methylphenyl)sulfonyl]-5-[({[(4-methylphenyl)sulfonyl]amino}carbonyl)amino]-1,3-dihydro-2H-isoindole-2-carboxamide (153.6 mg, 12%). NMR (300 MHz, d6-DMSO) δ 8.77 (1H, br s, NH), 7.79 (4H, dd, ArH), 7.54 (1H, d, ArH), 7.35 (4H, d, ArH), 7.18 (1H, d, ArH), 7.14 (1H, s, ArH), 4.63 (2H, br s, CH2), 4.40 (2H, br s, CH2), 2.35 (6H, s, 2×CH3). LCMS [M+H]+=529, TLC Rf=0.38 (10% MeOH:DCM).

### Example 15

### N-[(4-Methylphenyl)sulfonyl]-7-({[(4-methylphenyl)sulfonyl]carbamoyl}amino)-1,2,4,5-tetrahydro-3H-3-benzazepine-3-carboxamide

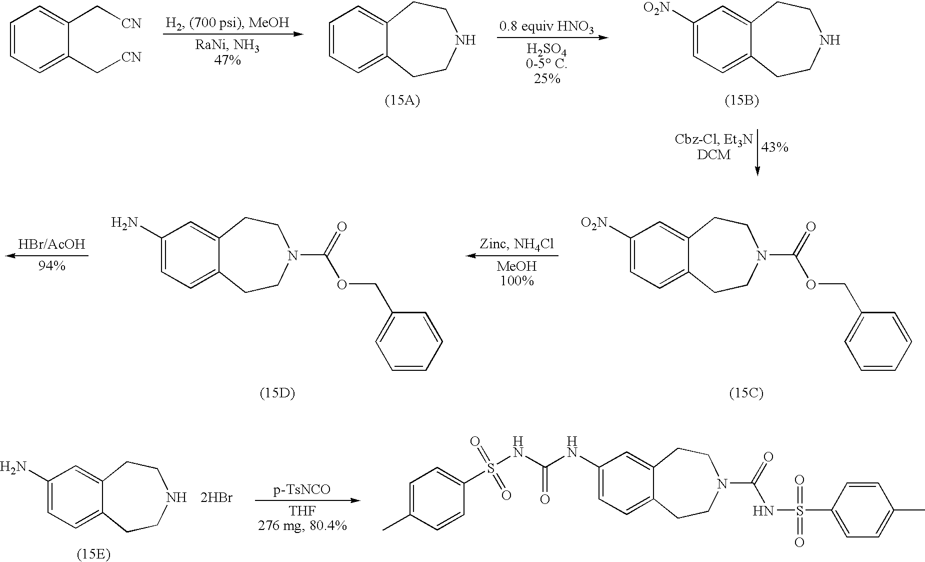

A mixture of 1,2-phenylenediacetonitrile (20 g, 0.128 mol) and Raney Nickel (2 g) in 20% methanolic ammonia (120 ml) and methanol (400 ml) were charged with hydrogen (700 psi). The mixture was then heated at 70° C. for 2 days. After cooling the mixture was filtered through celite and evaporated in vacuo. The resultant crude material was purified by dry flash column chromatography (eluting with 10% MeOH in DCM and 1% triethylamine) to yield 2,3,4,5-tetrahydro-1H-3-benzazepine as a brown oil (15A) (9 g, 47%).

¹H NMR (CDCl₃) 8.08-7.15 (4H, m, ArH), 2.96-2.86 (8H, m, 8H) ppm.

2,3,4,5-tetrahydro-1H-3-benzazepine (15A) (18.34 g, 0.124 mol) was cooled to 0° C. and conc. H₂SO₄ (70 ml) added. The mixture was stirred for 30 mins before conc. HNO₃ (4.55 ml, 0.099 mol) was added dropwise. The mixture was stirred for 2 hours at 0° C. before being poured onto ice/water (500 ml). The mixture was basified with solid NaOH and organics extracted with ethyl acetate (3×50 ml). The solvent was evaporated in vacuo to yield 7-nitro-2,3,4,5-tetrahydro-1H-3-benzazepine as a brown oil (15B) (6.07 g, 25%). This crude material was used directly in the next step. A small amount of material was purified by flash column chromatography (eluting with 1-10% MeOH/NH₃ in DCM) for characterisation.

¹H NMR (CDCl₃) 7.95-7.90 (2H, m, ArH), 7.25 (1H, m, ArH), 3.0-2.9 (8H, m) ppm.

LCMS [M+H]⁺ 193.

To a stirred mixture of 7-nitro-2,3,4,5-tetrahydro-1H-3-benzazepine (15B) (6.26 g, 32.5 mmol) and triethylamine (6.5 ml, 48.8 mmol) in DCM (100 ml) at 0° C. was added benzyl chloroformate (5.6 ml, 39.1 mmol). The mixture was allowed to warm to room temperature and stirred overnight. Water (80 ml) was then added and after stirring for 15 mins the organic layer was separated, dried (MgSO₄) and evaporated in vacuo. The resultant oil was purified by flash column chromatography (eluting with 4:1 hexane:ethyl acetate) to yield benzyl 7-nitro-1,2,4,5-tetrahydro-3H-3-benzazepine-3-carboxylate (15C) as a white solid (5.42 g, 43%).

¹H NMR (CDCl₃) 8.0 (2H, d, ArH), 7.4-7.35 (6H, m, ArH), 5.2 (2H, s, CH₂), 3.65 (4H, br s, 2×CH₂), 3.0 (4H, br s, 2×CH₂) ppm.

LCMS [M+H]⁺ 327.

Zinc dust (12.67 g, 13.85 mmol) was added to a mixture of benzyl 7-nitro-1,2,4,5-tetrahydro-3H-3-benzazepine-3-carboxylate (15C) (4.52 g, 13.85 mmol) and ammonium chloride (1.48 g, 27.70 mmol) in methanol (125 ml). The mixture was refluxed for 2 hours until tlc indicated no starting material was present. The mixture was cooled, filtered through celite and solvent evaporated in vacuo to yield benzyl 7-amino-1,2,4,5-tetrahydro-3H-3-benzazepine-3-carboxylate (15D) as an off-yellow solid (4.12, 100%).

¹H NMR (DMSO-d₆) 7.38-7.28 (5H, m, ArH), 6.75 (1H, d, ArH), 6.32 (1H, d, ArH), 6.28 (1H, dd, ArH), 5.1 (2H, s, CH₂), 4.82 (2H, s, NH₂), 3.45 (4H, br s, 2×CH₂), 2.64 (4H, br s, 2×CH₂) ppm.

LCMS [M+H]⁺ 297.

HBr in AcOH (50 ml) was added to benzyl 7-amino-1,2,4,5-tetrahydro-3H-3-benzazepine-3-carboxylate (15D) (4.12 g, 0.013 mol) and the mixture stirred for 3 hours at room temperature. The resultant yellow solid was washed with diethyl ether and dried to yield 2,3,4,5-tetrahydro-1H-3-benzazepin-7-amine 2HBr salt (15E) as an off-yellow solid (4.25 g, 94%).

¹H NMR (DMSO-d₆) 8.9 (2H, br s, NH₂), 7.26 (1H, m, ArH), 7.15 (2H, m, ArH), 3.1 (4H, br s, 2×CH₂), 3.05 (4H, br s, 2×CH₂) ppm.

LCMS [M+H]⁺ 163.

2,3,4,5-Tetrahydro-1H-3-benzazepin-7-amine 2HBr salt (15E) (100 mg, 0.308 mmol) was stirred in DCM (10 ml) and 2M NaOH (2 ml) was added. After stirring for 10 mins the DCM layer was separated and evaporated in vacuo. The free base was re-suspended in THF (8 ml) and p-toluenesulfonyl isocyanate was then added. After 2 hours stirring at room temperature, diethyl ether (˜15 ml) was added dropwise. The resultant solid was filtered, washed with small volumes of ether and dried to yield the title compound N-[(4-methylphenyl)sulfonyl]-7-({[(4-methylphenyl)sulfonyl]carbamoyl}amino)-1,2,4,5-tetrahydro-3H-3-benzazepine-3-carboxamide as a white solid (276 mg, 80%).

¹H NMR (DMSO-d₆) 10.8 (1H, br s, NH), 8.65 (1H, s, NH), 8.8-7.7 (4H, m, ArH), 7.4-7.32 (4H, m, ArH), 7.1-6.95 (3H, m, ArH), 3.35 (4H, br s, 2×CH₂), 2.65 (4H, br s, 2×CH₂), 2.36 (3H, s, CH₃), 3.34 (3H, s, CH₃) ppm.

LCMS [M+H]⁺ 557.

### Example 16

### N-Phenylsulfonyl-7-{[(phenylsulfonyl)carbamoyl]amino}-1,2,4,5-tetrahydro-3H-3-benzazepine-3-carboxamide

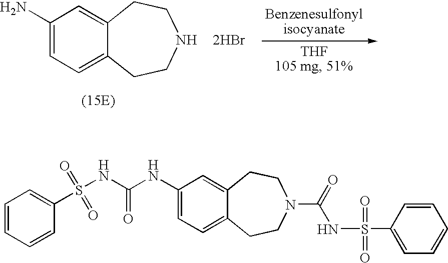

2,3,4,5-Tetrahydro-1H-3-benzazepin-7-amine 2HBr salt (15E) (100 mg, 0.308 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (2 ml) and benzenesulfonyl isocyanate was then added dropwise. After 2 hours stirring at room temperature, diethyl ether (˜15 ml) was added dropwise. The resultant solid was filtered, washed with small volumes of ether and dried to yield the title compound N-phenylsulfonyl-7-{[(phenylsulfonyl)carbamoyl]amino}-1,2,4,5-tetrahydro-3H-3-benzazepine-3-carboxamide as a white solid (105 mg, 51%).

¹H NMR (DMSO-d₆) 11.05 (1H, br s, NH), 8.55 (1H, s, NH), 7.92 (1H, d, ArH), 7.84 (1H, d, ArH), 7.56-7.28 (6H, m, ArH), 7.08-6.98 (3H, m, ArH), 3.58 (4H, br s, 2×CH₂), 2.68 (4H, br s, 2×CH₂), 2.58 (3H, s, CH₃), 2.52 (3H, s, CH₃) ppm.

LCMS [M+H]⁺ 557.

### Example 17

### N-[(3-Methylphenyl)sulfonyl]-7-({[(3-methylphenyl)sulfonyl]carbamoyl}amino)-1,2,4,5-tetrahydro-3H-3-benzazepine-3-carboxamide

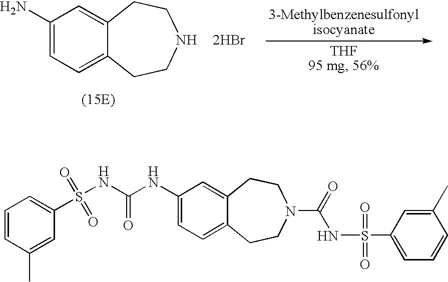

2,3,4,5-Tetrahydro-1H-3-benzazepin-7-amine 2HBr salt (15E) (100 mg, 0.308 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (3 ml) and m-toluenesulfonyl isocyanate (131 mg, 0.665 mmol) was then added dropwise. After 2 hours stirring at room temperature, diethyl ether (˜15 ml) was added dropwise. The resultant solid was filtered, washed with small volumes of ether and dried to yield the title compound N-[(3-methylphenyl)sulfonyl]-7-({[(3-methylphenyl)sulfonyl]carbamoyl}amino)-1,2,4,5-tetrahydro-3H-3-benzazepine-3-carboxamide as a white solid (95 mg, 56%).

¹H NMR (DMSO-d₆) 10.85 (1H, br s, NH), 8.72 (1H, s, NH), 7.74 (2H, m, ArH), 7.68 (2H, m, ArH), 7.49 (2H, d, ArH), 7.42 (2H, d, ArH), 7.1 (1H, s, ArH), 7.04 (2H, m, ArH), 3.3 (4H, br s, 2×CH₂), 2.7 (4H, br s, 2×CH₂), 2.36 (3H, s, CH₃), 2.34 (3H, S, CH₃) ppm.

LCMS [M+H]⁺ 557.

### Example 18

### N-[(2-Chlorophenyl)sulfonyl]-7-({[(2-chlorophenyl)sulfonyl]carbamoyl}amino)-1,2,4,5-tetrahydro-3H-3-benzazepine-3-carboxamide

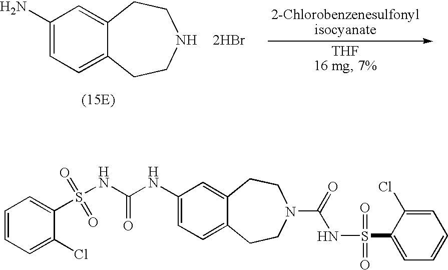

2,3,4,5-Tetrahydro-1H-3-benzazepin-7-amine 2HBr salt (15E) (100 mg, 0.308 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (3 ml) and o-chlorobenzenesulfonyl isocyanate (143 mg, 0.665 mmol) was then added dropwise. After 2 hours stirring at room temperature, diethyl ether (˜15 ml) was added dropwise. The resultant solid was filtered, washed with small volumes of ether and dried. This material was then further purified by SCX column chromatography eluting (with 8:2 DCM:MeOH) to yield N-[(2-chlorophenyl)sulfonyl]-7-({[(2-chlorophenyl)sulfonyl]carbamoyl}amino)-1,2,4,5-tetrahydro-3H-3-benzazepine-3-carboxamide as a white solid (16 mg, 7%).

¹H NMR (DMSO-d₆) 8.58 (1H, s, NH), 8.08 (1H, d, ArH), 7.98 (1H, d, ArH), 7.66 (6H, m, ArH), 7.8-7.0 (3H, m, ArH), 3.38 (4H, br s, 2×CH₂), 2.69 (4H, br s, 2×CH₂) ppm.

LCMS [M+H]⁺ 598.

### Example 19

### N-[(4-Chlorophenyl)sulfonyl]-7-({[(4-chlorophenyl)sulfonyl]carbamoyl}amino)-1,2,4,5-tetrahydro-3H-3-benzazepine-3-carboxamide

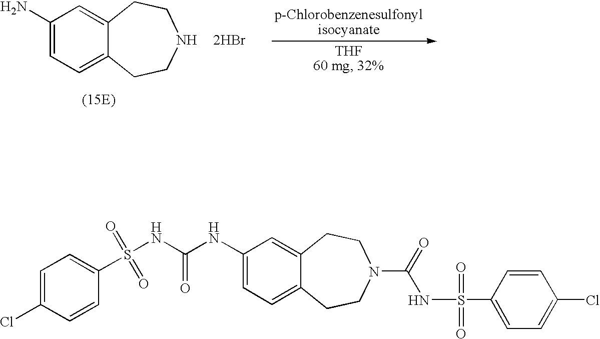

2,3,4,5-Tetrahydro-1H-3-benzazepin-7-amine 2HBr salt (100 mg, 0.308 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (3 ml) and p-chlorobenzenesulfonyl isocyanate (177 mg, 0.813 mmol) was then added dropwise. After 2 hours stirring at room temperature, diethyl ether (˜15 ml) was added dropwise. The resultant solid was filtered, washed with small volumes of ether and dried. This material was then further purified by SCX column chromatography (eluting with 8:2 DCM:MeOH) to yield N-[(4-chlorophenyl)sulfonyl]-7-({[(4-chlorophenyl)sulfonyl]carbamoyl}amino)-1,2,4,5-tetrahydro-3H-3-benzazepine-3-carboxamide as a white solid (60 mg, 33%).

¹H NMR (DMSO-d₆) 8.89 (1H, s, NH), 7.94 (2H, d, ArH), 7.84 (2H, d, ArH), 7.7 (4H, m, ArH), 7.10-7.0 (3H, m, ArH), 3.34 (4H, br s, 2×CH₂), 2.7 (4H, br s, 2×CH₂) ppm.

LCMS [M+H]⁺ 598.

### Example 20

### N-[(4-Fluorophenyl)sulfonyl]-7-({[(4-fluorophenyl)sulfonyl]carbamoyl}amino)-1,2,4,5-tetrahydro-3H-3-benzazepine-3-carboxamide

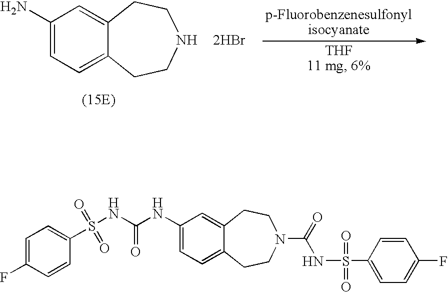

2,3,4,5-Tetrahydro-1H-3-benzazepin-7-amine 2HBr salt (100 mg, 0.308 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (3 ml) and p-fluorobenzenesulfonyl isocyanate (188 mg, 0.937 mmol) was then added dropwise. After 2 hours stirring at room temperature, diethyl ether (˜15 ml) was added dropwise. The resultant solid was filtered, washed with small volumes of ether and dried. This material was then further purified by SCX column chromatography (eluting with 8:2 DCM:MeOH) to yield N-[(4-fluorophenyl)sulfonyl]-7-({[(4-fluorophenyl)sulfonyl]carbamoyl}amino)-1,2,4,5-tetrahydro-3H-3-benzazepine-3-carboxamide as a white solid (11 mg, 6%).

¹H NMR (DMSO-d₆) 11.0 (1H, br s, NH), 8.82 (1H, s, NH), 8.0-7.89 (4H, m, ArH), 7.42-7.36 (4H, m, ArH), 7.08-6.95 (3H, m, ArH), 3.3 (4H, br s, 2×CH₂), 2.7 (4H, br s, 2×CH₂) ppm.

LCMS [M+H]⁺ 565.

### Example 21

### N-[(4-Methoxyphenyl)sulfonyl]-7-({[(4-methoxyphenyl)sulfonyl]-carbamoyl}amino)-1,2,4,5-tetrahydro-3H-3-benzazepine-3-carboxamide

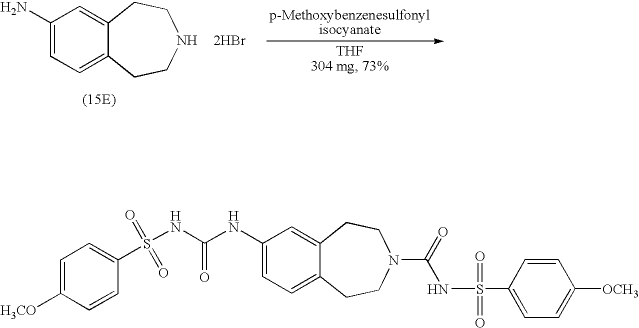

2,3,4,5-Tetrahydro-1H-3-benzazepin-7-amine 2HBr salt (200 mg, 0.616 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (3 ml) and p-methoxybenzenesulfonyl isocyanate (378 mg, 1.77 mmol) was then added dropwise. The mixture was stirred overnight and then diethyl ether (˜15 ml) was added dropwise. The resultant solid was filtered, washed with ether and dried to yield N-[(4-methoxyphenyl)sulfonyl]-7-({[(4-methoxyphenyl)sulfonyl]-carbamoyl}amino)-1,2,4,5-tetrahydro-3H-3-benzazepine-3-carboxamide as a white solid (304 mg, 73%).

¹H NMR (DMSO-d₆) 10.7 (1H, br s, NH), 8.62 (1H, s, NH), 7.85-7.78 (4H, m, ArH), 7.12-6.95 (7H, m, ArH), 3.82 (3H, s, OCH₃), 3.8 (3H, s, OCH₃), 3.34 (4H, br s, 2×CH₂), 2.65 (4H, br s, 2×CH₂) ppm.

LCMS [M+H]⁺ 589.

### Example 22

### N-[(4-tert-Butylphenyl)sulfonyl]-7-({[(4-tert-butylphenyl)sulfonyl]carbamoyl}-amino)-1,2,4,5-tetrahydro-3H-3-benzazepine-3-carboxamide

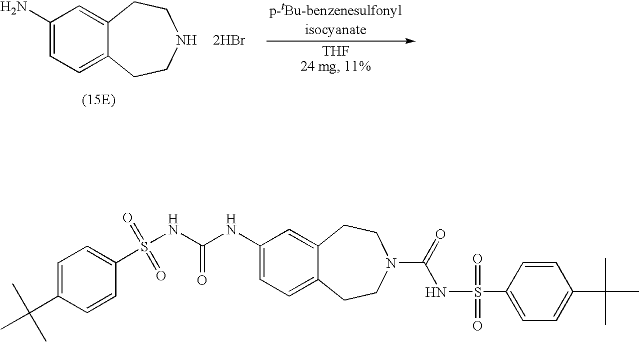

2,3,4,5-Tetrahydro-1H-3-benzazepin-7-amine 2HBr salt (100 mg, 0.308 mmol), prepared as described in Example 15 above, was stirred in DCM (10 ml) and 2M NaOH (2 ml) was added. After stirring for 10 mins the DCM layer was separated and evaporated in vacuo. The free base was re-suspended in THF (3 ml) and p-^{t}butylbenzenesulfonyl isocyanate (202 mg, 0.847 mmol) was then added dropwise. The mixture was stirred overnight and then diethyl ether (˜15 ml) was added dropwise. The resultant solid was then purified by SCX chromatography (eluting with 8:2 DCM:MeOH). This material was then dissolved in DCM (1 ml) and slowly triturated with diethyl ether. The solid was filtered and dried to yield N-[(4-tert-butylphenyl)sulfonyl]-7-({[(4-tert-butylphenyl)sulfonyl]carbamoyl}amino)-1,2,4,5-tetrahydro-3H-3-benzazepine-3-carboxamide as a white solid (24 mg, 11%).

¹H NMR (DMSO-d₆) 8.65 (1H, s, NH), 7.85 (2H, d, ArH), 7.78 (2H, d, ArH), 7.62-7.75 (4H, m, ArH), 7.1-6.98 (3H, m, ArH), 3.38 (4H, br s, 2×CH₂), 2.7 (4H, br s, 2×CH₂), 1.28 (9H, s, 3×CH₃), 1.27 (9H, s, 3×CH₃) ppm.

LCMS [M+H]⁺ 641.

### Example 23

### N-[(4-Methylphenyl)sulfonyl]-7-({[(4-methylphenyl)sulfonyl]carbamoyl}amino)-3,4-dihydroisoquinoline-2(1H)-carboxamide

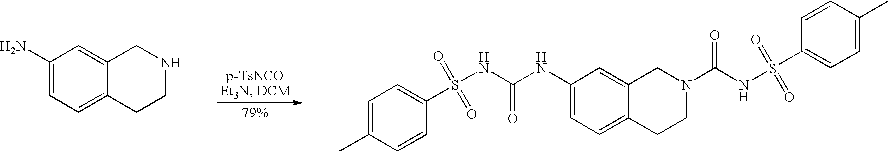

p-Toluenesulfonyl isocyanate (206 μL, 1.35 mmol) was added to a solution of 7-amino-1,2,3,4-tetrahydroisoquinoline (100 mg, 0.67 mmol) in DCM (1 ml) at room temperature. The mixture was stirred overnight and the solid that crashed out was filtered and washed with small volumes of diethyl ether to yield N-[(4-methylphenyl)sulfonyl]-7-({[(4-methylphenyl)sulfonyl]carbamoyl}-amino)-3,4-dihydroisoquinoline-2(1H)-carboxamide as a white solid (288 mg, 79%).

¹H NMR (DMSO-d₆) 11.12 (1H, br s, NH), 8.75 (1H, s, NH), 7.8-7.75 (4H, dd, ArH), 7.4-7.32 (4H, dd, ArH), 7.1-6.98 (3H, m, ArH), 4.35 (2H, br s, CH₂), 3.48 (2H, br s, CH₂), 2.62 (2H, t, CH₂), 2.38 (3H, s, CH₃), 2.36 (3H, s, CH₃) ppm.

LCMS [M+H]⁺ 543.

### Example 24

### N-[(4-Methylphenyl)sulfonyl]-7-([(4-methylphenyl)sulfonyl]amino)-1,2,4,5-tetrahydro-3H-3-benzazepine-3-carboxamide

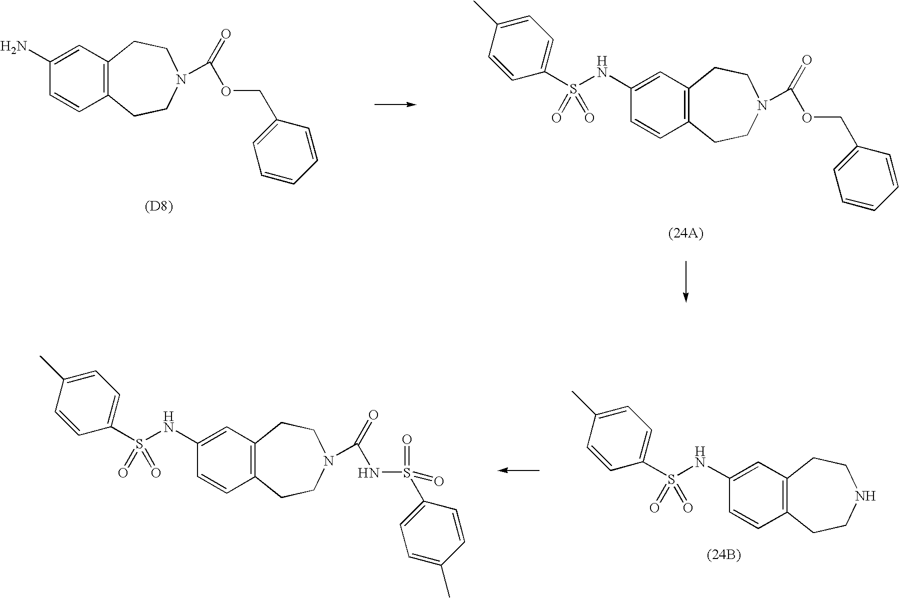

To a stirred mixture of benzyl 7-amino-1,2,4,5-tetrahydro-3H-3-benzazepine-3-carboxylate (D8) (200 mg, 0.67 mmol), prepared as described in Examples 6 to 10 above, and pyridine (140 μl, 1.35 mmol) in dichloromethane (10 ml) was added p-toluene sulfonyl chloride (135 mg, 1.01 mmol). The mixture was stirred overnight at room temperature. NaHCO₃ solution was added (5 ml) and after stirring for 15 mins the organic layer was separated, dried (MgSO₄) and evaporated in vacuo. The resultant crude material was purified by flash column chromatography (eluting with 8-60% gradient of EtOAc in Iso-hexane) to yield benzyl-7-([(4-methylphenyl)sulfonyl]amino)-1,2,4,5-tetrahydro-3H-3-benzazepine-3-carboxylate (24A) as a yellow oil (295 mg, 98%).

To a solution of benzyl-7-([(4-methylphenyl)sulfonyl]amino)-1,2,4,5-tetrahydro-3H-3-benzazepine-3-carboxylate (24A) (285 mg, 0.63 mmol) in methanol (30 ml) was added 10% Pd/C (50 mg). The resulting suspension was stirred under a H₂ balloon for 18 hours at room temperature. The catalyst was removed by filtration through celite and the resultant solution concentrated to yield 7-([(4-methylphenyl)sulfonyl]amino)-1,2,4,5-tetrahydro-3H-3-benzazepine (24B) as a yellow oil (170 mg, 85%).

To a stirred mixture of 7-([(4-methylphenyl)sulfonyl]amino)-1,2,4,5-tetrahydro-3H-3-benzazepine (24B) (85 mg, 0.17 mmol) and triethylamine (47 μl, 0.33 mmol) in dichloromethane (5 ml) was added p-toluenesulfonyl isocyanate (50 μl, 0.33 mmol). After 30 mins the reaction mixture was concentrated to give the crude product which was purified by preparative HPLC (Xbridge C-18 10*150 mm, 15 mins gradient 30-95%, 0.05% Formic Acid H₂O: 0.05% Formic Acid MeCN 10 ml/min) and the resulting fractions combined and freeze dried to give N-[(4-methylphenyl)sulfonyl]-7-([(4-methylphenyl)sulfonyl]amino)-1,2,4,5-tetrahydro-3H-3-benzazepine-3-carboxamide as white solid (40.3 mg, 46%).

LCMS [M+H]⁺ 514.

### Example 25

### N-[(4-Methylphenyl)sulfonyl]-7-([(4-biphenyl)sulfonyl]amino)-1,2,4,5-tetrahydro-3H-3-benzazepine-3-carboxamide

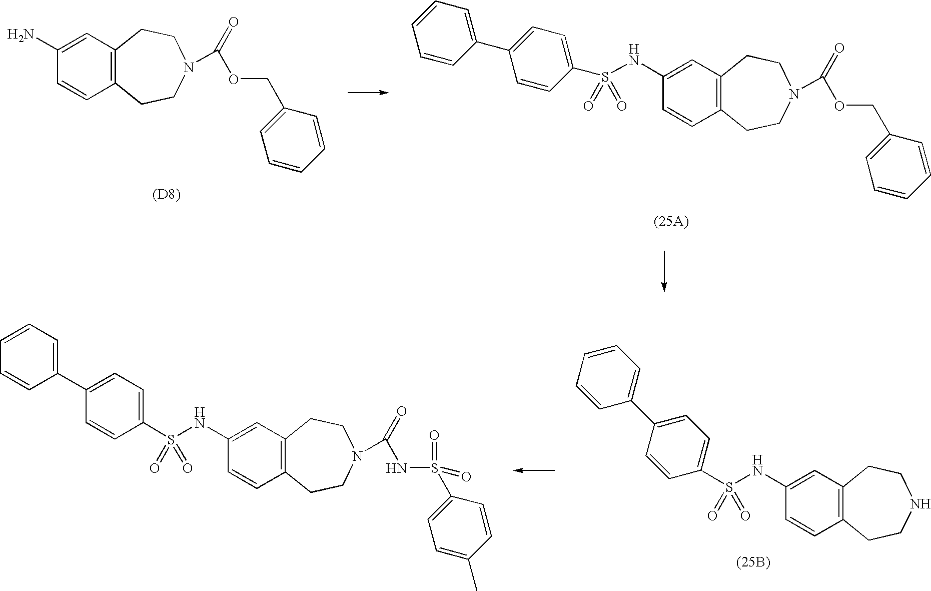

To a stirred mixture of benzyl 7-amino-1,2,4,5-tetrahydro-3H-3-benzazepine-3-carboxylate (D8) (100 mg, 0.34 mmol), prepared as described in Examples 6 to 10 above, and pyridine (5510.67 mmol) in dichloromethane (3 ml) was added biphenyl-4-sulfonyl chloride (128 mg, 0.51 mmol). The mixture was stirred overnight at room temperature and then washed with 1M HC (15 ml) the organic layer was separated, dried (MgSO₄) and evaporated in vacuo. The resultant crude material was purified by flash column chromatography (eluting with 8-60% gradient of EtOAc in iso-hexane) to yield benzyl-7-([(4-biphenyl)sulfonyl]amino)-1,2,4,5-tetrahydro-3H-3-benzazepine-3-carboxylate (25A) as a yellow oil (160 mg, 92%).

To a solution of benzyl-7-([(4-biphenyl)sulfonyl]amino)-1,2,4,5-tetrahydro-3H-3-benzazepine-3-carboxylate (25A) (160 mg, 0.31 mmol) in methanol (was added 10% Pd/C (30 mg). The resulting suspension was stirred under a H₂ balloon for 2 hours at room temperature. The catalyst was removed by filtration through celite and the resultant solution concentrated to yield 7-([(4-biphenyl)sulfonyl]amino)-1,2,4,5-tetrahydro-3H-3-benzazepine (25B) as a yellow oil (65 mg, 57%).

To a stirred mixture of 7-([(4-biphenyl)sulfonyl]amino)-1,2,4,5-tetrahydro-3H-3-benzazepine (25B) (65 mg, 0.17 mmol) and triethylamine (49 μl, 0.34 mmol) in a 1:1 solution of dichloromethane:tetrahydrofuran (10 ml) was added p-toluenesulfonyl isocyanate (52 μl, 0.34 mmol). After 30 mins the reaction mixture was concentrated to give the crude product which was purified by preparative HPLC (Xbridge C-18 10*150 mm, 15 mins gradient 30-95%, 0.05% Formic Acid H₂O: 0.05% Formic Acid MeCN 10 ml/min) and the resulting fractions combined and freeze dried to give N-[(4-methylphenyl)sulfonyl]-7-([(4-Biphenyl)sulfonyl]amino)-1,2,4,5-tetrahydro-3H-3-benzazepine-3-carboxamide as white solid (17.5 mg, 16%).

LC-MS [M+H]⁺ 575.

### Example 26

### N-[(4-Methylphenyl)sulfonyl]-7-([(4-methylphenyl)sulfonyl]amino)-1,2,4,5-tetrahydro-3H-3-benzazepine

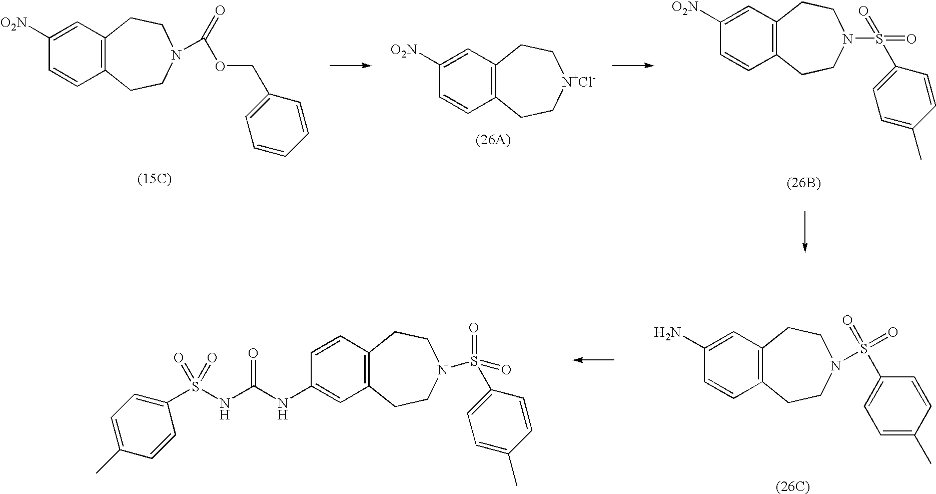

A stirred suspension of benzyl 7-nitro-1,2,4,5-tetrahydro-3H-3-benzazepine-3-carboxylate (15C) (200 mg, 0.61 mmol), prepared as described in Example 15, in 1M HCl (4 ml) in a sealed vessel was heated in a microwave at 160° C. for 30 mins. The resultant solution was concentrated to give the crude 7-nitro-1,2,4,5-tetrahydro-3H-3-benzazepine hydrochloride (26A) as a white solid which was used without any further purification.

To a stirred solution of crude 7-nitro-1,2,4,5-tetrahydro-3H-3-benzazepine hydrochloride (26A) (200 mg, 1.04 mmol) in dichloromethane (10 ml) was added pyridine (254 μl, 3.12 mmol), triethylamine (500 μl, 7.0 mmol) and p-toluene sulfonyl chloride. The reaction mixture was stirred at room temperature for 3 hours and concentrated in vacuo. The resultant crude material was purified by flash column chromatography (eluting with 8-60% gradient of EtOAc in Iso-hexane) to yield N-[(4-methylphenyl)sulfonyl]-7-nitro-1,2,4,5-tetrahydro-3H-3-benzazepine (26B) as a yellow oil (211 mg, 70%).

To a solution of N-[(4-methylphenyl)sulfonyl]-7-nitro-1,2,4,5-tetrahydro-3H-3-benzazepine (26B) (211 mg, 0.61 mmol) in tetrahydrofuran (10 ml) was added 10% Pd/C (30 mg). The resulting suspension was stirred under a H₂ balloon overnight at room temperature. The catalyst was removed by filtration through celite and the resultant solution concentrated to yield N-[(4-methylphenyl)sulfonyl]-7-amino-1,2,4,5-tetrahydro-3H-3-benzazepine (26C) as a white solid (170 mg, 88%).

To a stirred mixture of N-[(4-methylphenyl)sulfonyl]-7-amino-1,2,4,5-tetrahydro-3H-3-benzazepine (26C) (170 mg, 0.54 mmol) and triethylamine (153 μl, 1.07 mmol) in tetrahydrofuran (5 ml) was added p-toluenesulfonyl isocyanate (163 μl, 0.34 mmol). After 10 mins the reaction mixture was concentrated to give the crude product which was purified by preparative HPLC (Xbridge C-18 10*150 mm, 15 mins gradient 30-95%, 0.05% Formic Acid H₂O: 0.05% Formic Acid MeCN 10 ml/min) and the resulting fractions combined and freeze dried to give N-[(4-methylphenyl)sulfonyl]-7-([(4-methylphenyl)sulfonyl]amino)-1,2,4,5-tetrahydro-3H-3-benzazepine as white solid (46 mgs, 16%).

LC-MS [M+H]⁺ 514.

### Test Example 1

### Kv1.x T1

### Kvbeta Subunit Interaction Assays

Compounds of Examples 3, 13-15 and 18-22 and 24-26 were examined for their ability to inhibit the interaction between Kv1.x channels and Kvbeta subunits using a protein-protein interaction assay.

Cloning of rat His- and biotin-tagged Kvbeta1 subunit cDNA, expression of Kvbeta1 subunits in E. coli and lysis of E. coli containing Kvbeta1 subunits were carried out as previously described (Kozlowski et al., patent application WO03078464).

Cloning of His- and FLAG-tagged Kv1.1 alpha subunit T1 domain cDNA, expression of Kv1.1 alpha subunit T1 domains in E. coli and lysis of E. coli containing Kv1.1 alpha subunit T1 domains were carried out as previously described (Kozlowski et al., patent application WO03078464).

To 96-well streptavidin coated microtitre plates, 100 μl of optimized concentrations of Kvbeta1 subunit cleared lysates (typically 1 in 10 dilutions in PBS-Tween) were added to each well. Plates were incubated at room temperature for 1 hour prior to use. Each well was then washed 3 times with 300 μl of PBS-Tween. Following the final wash, 80 μl of PBS-Tween was added to each well.

The test compounds (dissolved in a suitable vehicle) were added to the wells of a prepared 96-well microliter plate to achieve a range of concentrations to maintain a final assay volume of 100 μl. Plates were incubated for 30 minutes at room temperature.

After incubation with test compounds, 100 μl of Kv1.1 alpha subunit T1 domain cleared lysate (diluted 1 in 10 in PBS-Tween) was added to each well and incubated for 60 minutes at room temperature on a shaker. Unbound Kv1.1 alpha subunit T1 domain was removed by washing 3 times in 300 μl of PBS-Tween. Bound Kv1.1 alpha subunit T1 domain was estimated by using appropriately diluted mouse anti-FLAG antibody and anti-mouse IgG secondary antibody HRP conjugate using standard ELISA procedures. HRP was detected as previously described (Kozlowski et al., patent application WO03078464).

Data were analyzed using standard software packages. The test compounds were examined for their ability to inhibit the binding of Kv1.1 alpha subunit T1 domain to Kvbeta1 subunits to determine inhibition as a percentage of maximal binding in the absence of any test compound. Results are presented as the half maximal inhibitory concentration (IC₅₀) for inhibition of Kv1.1 alpha subunit T1 binding to Kvbeta1 subunits. The tested compounds of this invention displayed the ability to inhibit the binding of Kv1.1 alpha subunit T1 domain to Kvbeta1 subunits as measured by determination of the IC₅₀ (Table 6).

TABLE 6

| Example | IC₅₀ (μM) |
|---|---|
| 3 | 50 |
| 13 | 30 |
| 14 | 16 |
| 15 | 59 |
| 17 | 47 |
| 18 | 181 |
| 19 | 51 |
| 20 | 191 |
| 21 | 38 |
| 22 | 41 |
| 24 | 81 |
| 25 | 88 |
| 26 | 102 |

1-97. (canceled)

98. A compound selected from the group consisting of the group consisting of compounds of general formula (1a), compounds of general formula (1b), compounds of general formula (1c) and compounds of general formula (1d), and pharmacologically acceptable salts and prodrugs thereof: 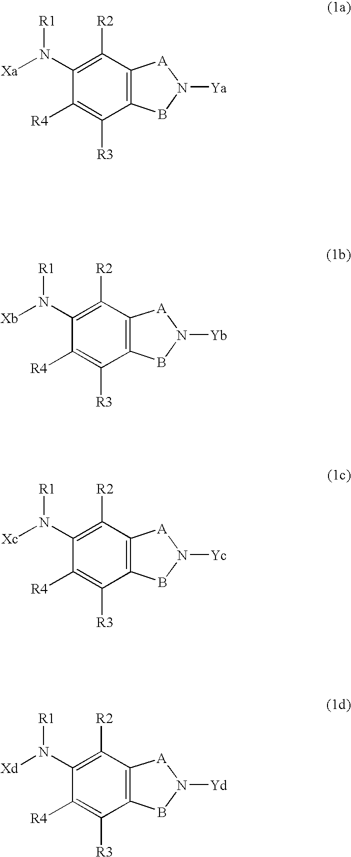
wherein:
A and B are independently CH₂ or CH₂CH₂;
R1 is a hydrogen atom, an alkyl group, a cycloalkyl group, an aryl group, an aralkyl group or a heteroaralkyl group;
R2, R3 and R4 are independently selected from hydrogen atoms, alkyl groups, halogen atoms, haloalkyl groups, alkoxy groups, alkoxycarbonyl groups, carboxyl groups, hydroxyl groups, nitro groups, amino groups, monoalkylamino groups, dialkylamino groups, acylamino groups, alkoxycarbonylamino groups, alkylsulphonyl groups, arylsulphonyl groups, aminosulphonyl groups and cyano groups;
Xa is R5aR7NSO₂ or R5aSO₂NR7CO, wherein R5a and R7 are as defined below;
Ya is selected from R6CO, R6SO₂, R6R7NCO, R6R7NSO₂, R6SO₂NR7CO and CO₂R8, wherein R6, R7 and R8 are as defined below;
R5a is selected from hydrogen atoms, alkyl groups,
aryl groups, aralkyl groups, heteroaryl groups and heteroaralkyl groups;
Xb is R5bCO, wherein R5b is a hydrogen atom, an alkyl group having from 1 to 4 carbon atoms or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
Yb is selected from R6SO₂, R6R7NSO₂, R6SO₂NR7CO, wherein R6 and R7 are as defined below;
Xc is R5cSO₂, wherein R5c is an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms;
Yc is selected from R6CO, R6SO₂, R6R7NSO₂ and R6SO₂NR7CO,
wherein R6 and R7 are as defined below;
Xd is CO₂R8, wherein R8 is as defined below;
Yd is selected from R6CO, R6SO₂, R6R7NCO, R6R7NSO₂ and
R6SO₂NR7CO, wherein R6 and R7 are as defined below;
R6 is selected from hydrogen atoms, alkyl groups,
aryl groups, aralkyl groups, heteroaryl groups and heteroaralkyl groups;
R7 is a hydrogen atom, an alkyl group, an aryl group or an aralkyl group; and
R8 is an alkyl group, an aryl group, an aralkyl group, an alkoxyalkyl group, a heteroaryl group or a heteroarylalkyl group.

99. A compound according to claim 98 or a pharmacologically acceptable salt or prodrug thereof wherein R1 is a hydrogen atom, an alkyl group having from 1 to 6 carbon atoms or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups.

100. A compound according to claim 98 or a pharmacologically acceptable salt or prodrug thereof wherein R2, R3 and R4 are independently selected from hydrogen atoms, alkyl groups having from 1 to 6 carbon atoms, halogen atoms, haloalkyl groups having from 1 to 6 carbon atoms, alkoxy groups having from 1 to 6 carbon atoms, alkoxycarbonyl groups comprising carbonyl groups substituted with an alkoxy group having from 1 to 6 carbon atoms, carboxy groups, hydroxyl groups and cyano groups.

101. A compound of general formula (1a) according to claim 98 or a pharmacologically acceptable salt or prodrug thereof wherein Xa is a group of formula R5aSO₂R7NCO or R5aR7NSO₂, wherein
R5a is: a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms, and
R7 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups.

102. A compound of general formula (1a) according to claim 98 or a pharmacologically acceptable salt or prodrug thereof wherein Xa is a group of formula R5aSO₂R7NCO or R5aR7NSO₂, wherein
R5a is an aryl group having from 6 to 10 carbon atoms which may optionally be substituted with at least one substituent selected from alkyl groups having from 1 to 4 carbon atoms, halogen atoms, alkyoxy groups having from 1 to 4 carbon atoms, phenyl groups and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, and
R7 is a hydrogen atom or an alkyl group having from 1 to 4 carbon atoms.

103. A compound of general formula (1b) according to claim 98 or a pharmacologically acceptable salt or prodrug thereof wherein Xb is a group of formula R5bCO, wherein R5b is: a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms.

104. A compound general formula (1b) according to claim 98 or a pharmacologically acceptable salt or prodrug thereof wherein Xb is a group of formula R5bCO, wherein R5b is a hydrogen atom; an alkyl group having from 1 to 4 carbon atoms or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms.

105. A compound of general formula (1c) according to claim 98 or a pharmacologically acceptable salt or prodrug thereof wherein Xc is a group of formula R5cSO₂, wherein R5c is: a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms.

106. A compound of general formula (1d) according to claim 98 or a pharmacologically acceptable salt or prodrug thereof wherein X is a group of formula CO₂R8 wherein R8 is: an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups.

107. A compound of general formula (1a), (1c) or (1d) according to claim 98 or a pharmacologically acceptable salt or prodrug thereof wherein Ya, Yc or Yd is a group of formula R6CO, wherein R6 is a hydrogen atom, an alkyl group having from 1 to 6 carbon atoms; an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms.

108. A compound according to claim 98 or a pharmacologically acceptable salt or prodrug thereof wherein Ya, Yb, Yc or Yd is a group of formula R6SO₂, wherein R6 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms.

109. A compound of general formula (1a) or (1d) according to claim 98 or a pharmacologically acceptable salt or prodrug thereof wherein Ya or Yd is a group of formula R6R7NCO, wherein
R6 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms, and
R7 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups.

110. A compound according to claim 98 or a pharmacologically acceptable salt or prodrug thereof wherein Ya, Yb, Yc or Yd is a group of formula R6SO₂R7NCO, wherein R6 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, a heteroaryl group which is a 5- to 7-membered aromatic heterocyclic group containing 1 to 3 sulfur atoms, oxygen atoms and/or nitrogen atoms, or a heteroaralkyl group which comprises an alkyl group having from 1 to 6 carbon atoms which is substituted with a heteroaryl group which is a 5- to 7-membered aromatic heterocyclic group containing 1 to 3 sulfur atoms, oxygen atoms and/or nitrogen atoms, and
R7 is a hydrogen atom, an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups.

111. A compound of general formula (1a) according to claim 98 or a pharmacologically acceptable salt or prodrug thereof wherein Ya is a group of formula CO₂R8 wherein R8 is an alkyl group having from 1 to 6 carbon atoms, an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, phenyl groups, alkoxy groups having from 1 to 6 carbon atoms, alkoxycarbonyl groups wherein the alkoxy group has from 1 to 6 carbon atoms, carboxyl groups, hydroxyl groups and cyano groups, or an aralkyl group comprising an alkyl group having from 1 to 6 carbon atoms which is substituted with an aryl group having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups and cyano groups.

112. A compound of general formula (1a) according to claim 98 or a pharmacologically acceptable salt or prodrug thereof wherein:
each of R1, R2, R3 and R4 is a hydrogen atom;
Xa is a group of formula R5aSO₂R7NCO, wherein R5a is a phenyl group which may optionally be substituted with a methyl group, a t-butyl group, a fluorine atom, a chlorine atom, a methoxy group or a phenyl group and R7 is a hydrogen atom; and
Ya is a group of formula R6CO (wherein R6 is a hydrogen atom; a methyl group, a benzyl group or a phenethyl group), a group of formula R6SO₂ (wherein R6 is a phenyl group which may optionally be substituted with at least one substituent selected from methyl groups, ethyl groups, chlorine atoms and fluorine atoms) or a group of formula R6R7NCO (wherein R5 is a hydrogen atom, a benzyl group or a phenyl group which is optionally substituted with a methyl group and R7 is a hydrogen atom).

113. A compound of general formula (1b) according to claim 98 or a pharmacologically acceptable salt or prodrug thereof wherein:
each of R1, R2, R3 and R4 is a hydrogen atom;
Xb is a group of formula R5bCO, wherein R5b is a hydrogen atom, a methyl group, a benzyl group or a phenethyl group; and
Yb is a group of formula R6bSO₂R7NCO, wherein R6b is a phenyl group which may optionally be substituted with a methyl group, a t-butyl group, a fluorine atom, a chlorine atom or a methoxy group and R7 is a hydrogen atom.

114. A compound of general formula (1c) according to claim 98 or a pharmacologically acceptable salt or prodrug thereof wherein:
each of R1, R2, R3 and R4 is a hydrogen atom;
Xc is a group of formula R5SO₂ (wherein R5 is a phenyl group which may optionally be substituted with at least one substituent selected from methyl groups, ethyl groups, chlorine atoms and fluorine atoms); and
Yc is a group of formula R6SO₂R7NCO (wherein R6 is a phenyl group which may optionally be substituted with a methyl group, a t-butyl group, a fluorine atom, a chlorine atom or a methoxy group and R7 is a hydrogen atom).

115. A compound of general formula (1d) according to claim 98 or a pharmacologically acceptable salt or prodrug thereof wherein:
each of R1, R2, R3 and R4 is a hydrogen atom;
Xd is a group of formula CO₂R8 wherein R8 is an alkyl group having from 1 to 4 carbon atoms, an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms, or an aralkyl group comprising an alkyl group having from 1 to 4 carbon atoms which is substituted with an aryl group having from 6 to 10 carbon atoms which may optionally be substituted with at least one substituent selected from alkyl groups having from 1 to 4 carbon atoms, halogen atoms and haloalkyl groups having from 1 to 4 carbon atoms; and
Yc is a group of formula R6SO₂R7NCO (wherein R6 is a phenyl group which may optionally be substituted with a methyl group, a t-butyl group, a fluorine atom, a chlorine atom, a methoxy group or a phenyl group and R7 is a hydrogen atom).

116. compound of general formula (1a) according to claim 98 or a pharmacologically acceptable salt or prodrug thereof wherein:
each of R1, R2, R3 and R4 is a hydrogen atom;
Ya is a group of formula R6CO, wherein R6 is a hydrogen atom, a methyl group, a benzyl group or a phenethyl group; and
Xa is a group of formula R5aSO₂R7NCO, wherein R5a is a phenyl group which may optionally be substituted with a methyl group, a t-butyl group, a fluorine atom, a chlorine atom, a methoxy group or a phenyl group and R7 is a hydrogen atom.

117. A compound of general formula (1a) according to claim 98 or a pharmacologically acceptable salt or prodrug thereof wherein:
each of R1, R2, R3 and R4 is a hydrogen atom;
Ya is a group of formula R6SO₂ (wherein R6 is a phenyl group which may optionally be substituted with at least one substituent selected from methyl groups, ethyl groups, chlorine atoms and fluorine atoms); and
Xa is a group of formula R5aSO₂R7NCO (wherein R5a is a phenyl group which may optionally be substituted with a methyl group, a t-butyl group, a fluorine atom, a chlorine atom, a methoxy group or a phenyl group and R7 is a hydrogen atom).

118. A compound of general formula (1a) according to claim 98 or a pharmacologically acceptable salt or prodrug thereof wherein:
each of R1, R2, R3 and R4 is a hydrogen atom;
Ya is a group of formula R6R7NCO (wherein R6 is a hydrogen atom, a benzyl group or a phenyl group which is optionally substituted with a methyl group, and R7 is a hydrogen atom); and
Xa is a group of formula R5aSO₂R7NCO (wherein R5a is a phenyl group which may optionally be substituted with a methyl group, a t-butyl group, a fluorine atom, a chlorine atom, a methoxy group or a phenyl group and R7 is a hydrogen atom).

119. A compound of general formula (1b) according to claim 98 or a pharmacologically acceptable salt or prodrug thereof wherein:
each of R1, R2, R3 and R4 is a hydrogen atom;
Yb is a group of formula R6SO₂R7NCO, wherein R6 is a phenyl group which may optionally be substituted with a methyl group, and R7 is a hydrogen atom; and either
Xb is a group of formula R5bCO (wherein R5b is a hydrogen atom; a methyl group, a benzyl group or a phenethyl group), or
Xc is a group of formula R5cSO₂ (wherein R5c is a phenyl group which may optionally be substituted with at least one substituent selected from methyl groups, ethyl groups, chlorine atoms and fluorine atoms).

120. A compound according to claim 98 or a pharmacologically acceptable salt or prodrug thereof selected from the group consisting of:
N-phenylsulfonyl-7-[({[(4-methylphenyl)sulfonyl]amino}carbonyl)amino]-1,2,4,5-tetrahydro-3H-3-benzazepine-3-carboxamide;
N-acetyl-7-[({[(4-methylphenyl)sulfonyl]amino}carbonyl)amino]-1,2,4,5-tetrahydro-3H-3-benzazepine-3-carboxamide;
N-benzylaminocarbonyl-7-[({[(4-methylphenyl)sulfonyl]amino}-carbonyl)amino]-1,2,4,5-tetrahydro-3H-3-benzazepine-3-carboxamide;
N-(1-oxo-3-phenyl-propanyl)-7-[({[(4-methylphenyl)sulfonyl]amino}-carbonyl)amino]-1,2,4,5-tetrahydro-3H-3-benzazepine-3-carboxamide;
N-formyl-7-[({[(4-methylphenyl)sulfonyl]amino}carbonyl)amino]-1,2,4,5-tetrahydro-3H-3-benzazepine-3-carboxamide;
N-[(4-methylphenyl)sulfonyl]-7-phenylsulfonylamino-1,2,4,5-tetrahydro-3H-3-benzazepine-3-carboxamide;
N-[(4-methylphenyl)sulfonyl]-7-acetylamino-1,2,4,5-tetrahydro-3H-3-benzazepine-3-carboxamide;
N-[(4-methylphenyl)sulfonyl]-7-benzylaminocarbonylamino-1,2,4,5-tetrahydro-3H-3-benzazepine-3-carboxamide;
N-[(4-methylphenyl)sulfonyl]-7-(1-oxo-3-phenyl-propanylamino)-1,2,4,5-tetrahydro-3H-3-benzazepine-3-carboxamide;
N-[(4-methylphenyl)sulfonyl]-7-formylamino-1,2,4,5-tetrahydro-3H-3-benzazepine-3-carboxamide;
7-[({[(4-methylphenyl)sulfonyl]amino}carbonyl)amino]-1,2,4,5-tetrahydro-3H-3-benzazepine-3-carboxamide;
N-phenylsulfonyl-7-phenylsulfonylaminocarbonylamino-1,2,4,5-tetrahydro-3H-3-benzazepine-3-carboxamide;
N-[(4-methylphenyl)sulfonyl]-6-({[(4-methylphenyl)sulfonyl]-carbamoyl}amino)-3,4-dihydroisoquinoline-2(1H)-carboxamide;
N-[(4-methylphenyl)sulfonyl]-5-[({[(4-methylphenyl)sulfonyl]amino}carbonyl)amino]-1,3-dihydro-2H-isoindole-2-carboxamide;
N-[(4-methylphenyl)sulfonyl]-7-({[(4-methylphenyl)sulfonyl]carbamoyl}amino)-1,2,4,5-tetrahydro-3H-3-benzazepine-3-carboxamide;
N-phenylsulfonyl-7-{[(phenylsulfonyl)carbamoyl]amino}-1,2,4,5-tetrahydro-3H-3-benzazepine-3-carboxamide;
N-[(3-methylphenyl)sulfonyl]-7-({[(3-methylphenyl)sulfonyl]carbamoyl}amino)-1,2,4,5-tetrahydro-3H-3-benzazepine-3-carboxamide;
N-[(2-chlorophenyl)sulfonyl]-7-({[(2-chlorophenyl)sulfonyl]carbamoyl}amino)-1,2,4,5-tetrahydro-3H-3-benzazepine-3-carboxamide;
N-[(4-chlorophenyl)sulfonyl]-7-({[(4-chlorophenyl)sulfonyl]carbamoyl}amino)-1,2,4,5-tetrahydro-3H-3-benzazepine-3-carboxamide;
N-[(4-fluorophenyl)sulfonyl]-7-({[(4-fluorophenyl)sulfonyl]carbamoyl}amino)-1,2,4,5-tetrahydro-3H-3-benzazepine-3-carboxamide;
N-[(4-methoxyphenyl)sulfonyl]-7-({[(4-methoxyphenyl)sulfonyl]carbamoyl}amino)-1,2,4,5-tetrahydro-3H-3-benzazepine-3-carboxamide;
N-[(4-tert-butylphenyl)sulfonyl]-7-({[(4-tert-butylphenyl)sulfonyl]carbamoyl}amino)-1,2,4,5-tetrahydro-3H-3-benzazepine-3-carboxamide;
N-[(4-methylphenyl)sulfonyl]-7-({[(4-methylphenyl)sulfonyl]carbamoyl}amino)-3,4-dihydroisoquinoline-2(1H)-carboxamide;
N-[(4-methylphenyl)sulfonyl]-7-([(4-methylphenyl)sulfonyl]amino)-1,2,4,5-tetrahydro-3H-3-benzazepine-3-carboxamide;
N-[(4-methylphenyl)sulfonyl]-7-([(4-biphenyl)sulfonyl]amino)-1,2,4,5-tetrahydro-3H-3-benzazepine-3-carboxamide; and
N-[(4-methylphenyl)sulfonyl]-7-([(4-methylphenyl)phenyl)sulfonyl]-amino)-1,2,4,5-tetrahydro-3H-3-benzazepine.

121. A pharmaceutical composition comprising a pharmacologically acceptable diluent or carrier and an active ingredient, wherein said active ingredient is a compound according to claim 98 or a pharmacologically acceptable salt or prodrug thereof as an active ingredient thereof.

122. A compound according to claim 98 or a pharmacologically acceptable salt or prodrug thereof for use as a medicament.

123. A method for the prophylaxis or treatment of a disease selected from the group consisting of diseases in which Kv1.x channels are involved, diseases ameliorated by Kv1.x channel opening, conditions or diseases ameliorated by Kv1.x channel inhibition, lower urinary tract disorders, anxiety and anxiety-related conditions, epilepsy, pain disorders, gynecological pain, cardiac arrhythmias, thromboembolic events, cardiovascular diseases, disorders of the auditory system, migraine, gastrointestinal disorders, vascular and visceral smooth muscle disorders, cell proliferative disorders, metabolic disorders, memory loss, CNS-mediated motor dysfunction disorders and ophthalmic disorders, said method comprising administering to a patient in need thereof an effective amount of a compound according to claim 98 or a pharmacologically acceptable salt or prodrug thereof.

124. A pharmaceutical composition comprising a pharmacologically acceptable diluent or carrier and a combination of active ingredients, wherein said active ingredients comprise at least one compound according to claim 98 or a pharmacologically acceptable salt or prodrug thereof in combination at least one compound selected from the group consisting of muscarinic receptor antagonists, β3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists and alpha-1 adrenoceptor antagonists.

125. A pharmaceutical composition comprising a pharmacologically acceptable diluent or carrier and a combination of active ingredients, wherein said active ingredients comprise at least one compound according to claim 98 or a pharmacologically acceptable salt or prodrug thereof in combination at least one compound selected from the group consisting of neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs).

126. A pharmaceutical composition comprising a pharmacologically acceptable diluent or carrier and a combination of active ingredients, wherein said active ingredients comprise at least one compound according to claim 98 or a pharmacologically acceptable salt or prodrug thereof in combination at least one compound selected from the group consisting of muscarinic receptor antagonists, β3 adrenergic receptor agonists, neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), 5-HT antagonists and alpha-1 adrenoceptor antagonists for use in the prophylaxis or treatment of lower urinary tract disorders.

127. A pharmaceutical composition comprising a pharmacologically acceptable diluent or carrier and a combination of active ingredients, wherein said active ingredients comprise at least one compound according to claim 98 or a pharmacologically acceptable salt or prodrug thereof in combination at least one compound selected from the group consisting of neurokinin K receptor antagonists, vanilloid VR1 agonists, calcium channel α2 δ ligands, potassium channel activators, calcium channel inhibitors, sodium channel blockers, serotonin and norepinephrine reuptake inhibitors (SNRIs), tricyclic antidepressants, N-methyl-D-aspartate (NMDA) receptor antagonists, cannabinoid receptor agonists, anti-convulsants, aldose reductase inhibitors, opioids, alpha adrenoceptor agonists, P2X receptor antagonists, acid-sensing ion channel modulators, NGF receptor modulators, nicotinic acetylcholine receptor modulators, synaptic vesicle protein 2A ligands and non-steroidal anti-inflammatory drugs (NSAIDs) for use in the prophylaxis or treatment of pain.
